(19) United States
(12) Patent Application Publication
(10) Pub. No.: US 2026/0077137 A1
(43) Pub. Date: Mar. 19, 2026

(54) INJECTION DEVICE

(71) Applicant: UNIVERSITÄT ZÜRICH, Zürich (CH)

(72) Inventors: Florian SCHMID, Zürich (CH); Beatrix RAUCH SCHMID, Luzern (CH); Rita PETTINELLO, Zürich (CH); Daniel EBERLI, Zürich (CH); Michael KUMMER, Zürich (CH); Ulrich Samuel Nino LEMANN, Zürich (CH); Philipp STÜCKLIN, Zürich (CH)

(21) Appl. No.: 19/106,907

(22) PCT Filed: Sep. 1, 2023
(86) PCT No.: PCT/EP2023/074044

§ 371 Date: Feb. 26, 2025

(30) Foreign Application Priority Data
Sep. 2, 2022 (EP) 22193690.9

(51) Int. Cl. A61M 5/34; A61M 5/315; A61M 5/42; A61M 5/44; A61M 25/01

(52) U.S. Cl. CPC A61M 5/344; A61M 5/31525; A61M 5/427; A61M 5/44; A61M 2005/31508; A61M 2025/0166; A61M 2205/3375

(57) ABSTRACT

Provided is an injection device for injection of a substance with an syringe into an organism, comprising a holding unit, which is adapted to hold the syringe, an actuating unit, which is adapted for dosed injection of the injection substance and which is removably coupled to the holding unit and couplable with the syringe when the holding unit holds the syringe, a needle guide which is adapted to be removably couplable with an injection needle and for changing the injection position of the injection needle; and a connection structure which connects the holding unit and the needle guide.

### FIELD OF THE INVENTION

The invention relates to an injection device which is arranged for positioning of an injection needle and dosed delivery of an injection substance, a medical device equipped with such an injection device, and a method for injection of an injection substance into biological tissue, in particular muscle tissue.

### BACKGROUND OF THE INVENTION

In recent years, several therapeutic approaches have been developed for the treatment of incontinence, like stress urinary incontinence (SUI), male stress urinary incontinence after prostatectomy, and fecal incontinence. SUI is characterized by involuntary loss of urine associated with exertion, effort, or coughing/sneezing. Several non-operative and operative options are available in the treatment of female SUI. Urethral injections are a well-described, minimally invasive technique and represent a common procedure for SUI. Several urethral bulking agents (UBAs) are available for injection as for example summarized in Li and Westney, Urol Clin North Am 46 (2019), 1-15, and recently cell therapy approaches were further developed, as for example described in WO 2019/215090 A1.

The injection of bulking agents and cells is typically carried out with an injection needle with simultaneous observation of the tissue, for example with an ultrasonic probe or a cystoscope. For practical medical applications, a precise positioning of the injection needle is of considerable importance, in order to deposit the injectable substance exactly at a desired injection site and to minimize the risk of damage to the surrounding tissue.

For positioning the injection needle, usually injection devices with a needle guide are used, which has a predetermined position and orientation relative to the ultrasonic probe; see for example WO 02/45588 A1.

Although some injection devices are known in the art, there is a constant need to provide devices which allow a precise injection of the injection substrate.

### DESCRIPTION OF THE INVENTION

The present invention provides an injection device which is provided for injection of an injection substance with a syringe into an organism and which is in particular suitable for injection into a sphincter muscle as well as a medical device, which comprises said injection device, and a method of directed injection, preferably of a defined volume.

The term “injection syringe” or “syringe” is generally used here to refer to any fluid delivery device having a fluid reservoir (syringe body with at least one chamber), as well as at least one syringe plunger including a piston and an outlet for the fluid. Preferably, the injection syringe further comprises an injection needle (hollow syringe needle). The syringe needle may be straight or curved. The syringe body may have one or more chambers, preferably cylindrical in shape. Each of the chambers is provided for receiving a syringe plunger including a piston. A displacement of the at least one syringe plunger in the syringe body may cause the injectable substance, e.g., a fluid to be expelled from the syringe body through the outlet, i.e., nozzle, preferably through a syringe needle which is connected to that outlet. For example, the syringe may comprise a syringe body having a single chamber to which the syringe needle is connected and in which a syringe plunger is disposed, or two or more chambers to which the syringe needle is connected and in each of which a syringe plunger is disposed.

The term “injection substance” is used here to refer generally to any injectable substance, i.e., a substance which can pass under pressure through a syringe. Preferably, the injectable substance is a liquid that contains at least one pharmacologically or biologically active substance in dissolved or suspended form. According to a preferred application of the invention, the injection substance comprises a cell suspension (or dispersion), in particular a suspension of muscle derived precursor cells. In particular, said cells are suspended in a suitable liquid, e.g., a buffer, cell medium, and/or carrier solution, like collagen. Such cells are for example described in WO 2019/216090 A1, which content is herein incorporated by reference.

With respect to the device, the present invention provides an injection device which comprises at least a holding unit for the syringe, i.e., it is designed to hold the syringe, an actuating unit, and a needle guide, and optionally a structure which connects at least the holding unit with the needle guide. The injection device of the present invention is designed for the injection of an injectable substance at a specific, preferably constant, dose, i.e. it is designed for dosed injection of the injection substance, and more particularly for repeated dosed injections of specified, preferably constant dose at a plurality of different injection sites.

Dosed injection is realized by an actuating unit as one part of the injection device of the present invention, which comprises a lever and a piston and which is removably coupled with the syringe in the holding unit. In particular, it is coupled with the plunger of the syringe. By operating the lever it is ensured that a specific/predefined amount of the injection substance, i.e. a specific dose, is dispensed. Accordingly, the actuating unit can also be referred to as dosing unit. The dosed injection is ensured by a predetermined displacement distance of the lever of the actuating unit which is translated via the piston of the actuating unit by coupling to the syringe plunger into a predetermined distance, by which the syringe plunger is moved into the syringe body resulting in the injection of a specific amount of the injectable substance through the needle.

In more detail, the actuating unit of the injection device of the present invention is removably coupled to the holding unit and is couplable with the syringe via its plunger when the holding unit holds the syringe, wherein the actuating unit comprises a lever and a piston, wherein a displacement of the lever from a first position to a second position, which is preferably performed by pivoting the lever from the first position to the second position about a pivot axis, results in a movement of the piston along its longitudinal axis, wherein the actuating unit is adapted to transfer the movement at least partly to the plunger of the syringe when the syringe is coupled with the actuating unit. In particular, the displacement of the lever results in an advancing movement of the piston along its longitudinal axis in a forward direction, i.e., towards the piston stop. The distance between the first and the second position of the lever is predetermined so that it is ensured that the piston advances by a predetermined distance. Accordingly, when the distance between the first and the second position of the lever is changed, it has an influence of the movement of the piston. In other words, the piston moves from a first position to a second position, wherein the distance is predetermined by the distance between the first and second position of the lever.

In even more detail, the actuating unit of the injection device of the present invention further comprises an actuator (clamping piece) through which the piston extends, and wherein the displacement of the lever from the first position to the second position causes the lever to engage the actuator, preferably via a pivot point in such a way that the actuator is frictionally connected with the piston, causing the actuator to advance together with the piston along the longitudinal axis in the first piston movement direction (forward direction), i.e. the piston of the actuating unit is moved towards the plunger of the syringe, when the syringe is placed in the holding unit and the actuating device is coupled with the syringe, in such a way that the plunger of the syringe is advanced in the first plunger movement direction (forward direction), i.e. the plunger of the syringe is moved so that the injection substance is dispensed from the syringe body. The frictional connection between the actuator and the piston of the actuating unit can be caused by (slightly) pivoting the actuator with respect to a perpendicular orientation of the actuator and the piston. Thus, when the lever is displaced from the first to the second position, the piston of the actuating unit is displaced along the longitudinal axis by a certain displacement distance, i.e. from a first position to a second position, in the direction of the piston stop (forward direction). In other words, tilting the actuator results in a frictional connection between the actuator and the piston rod by causing them to frictionally engage with each other, which then leads to the aforementioned advance movement. The advance movement of the piston exerts a force in the direction of the plunger of the syringe, resulting in a forward direction of said plunger, which results in metered substrate delivery. The displacement of the piston of the actuating unit is completed, i.e. the second position is reached, when the actuator and/or the lever arm abuts against the stop in the housing of the actuating unit. The actuation of the actuator is performed by spring force. The forward direction is understood as towards the nozzle of the syringe, wherein the position of the nozzle on the syringe is defined as at the front and the movement of an object, e.g., the injectable substance or the plunger through the syringe towards it as movement in forward direction, and the other open end of the syringe, through which the plunger is introduced into the syringe, is defined as the back end of the syringe and the movement of an object, e.g., the injectable substance or the plunger through the syringe towards the back end is defined as movement in the reverse or backward direction.

When the lever is displaced back into the first position, i.e. from the second to the first position, the connection between the actuator and the piston is released and the actuator can slide along the piston rod such that the actuator is displaced with respect to the piston rod from the second position to its first position.

Thus, when bringing the lever of the injection device of the present invention from the first to the second position, a specific amount of the injection substance, i.e. a specific dose, is dispensed, in particular dispensed through the nozzle of the syringe via a needle, and thus injected (if applied to an animal or human body) without the need to manually advance the plunger of the syringe along its longitudinal axis in a forward direction, the latter being less accurate as regards the dispensed amount of injection substance since the distance by which the plunger of the syringe is to be advanced to guarantee a certain amount of the injection substance to be dispensed has to be estimated by the operator, for example the physician, and is not predetermined by the predetermined displacement distance of the lever.

To ensure the injection of a specific dose without large variations is particularly important for clinical studies and later therapeutic approaches. For example, a correct dose-finding study is of the utmost importance during clinical development of a new drug. It must define the no-effective dose and the mean effective and maximal effective doses. Then taking tolerability into account, the optimal therapeutic dose range can be selected. Thus, when using the injection device of the present invention in clinical studies and therapeutic applications, the administration of an exact amount of injection substance is ensured.

In one embodiment, the injection device of the present invention, in particular the actuating unit of the injection device of the present invention further comprises a retaining element, preferably a retaining bracket (clamping plate), wherein the retaining element frictionally engages the piston rod in a retaining position in such a way that the piston is held in position and prevents the piston from being retracted along its longitudinal axis, i.e. moved backwards (opposite direction to the above-mentioned forward direction) when the lever and the actuator, respectively, move from the second position to the first position. This allows the re-actuation of the actuating unit, i.e. the movement of the lever (and the actuator) again from a first position to a second position, resulting in renewed metered substrate delivery. By actuating the retaining bracket, it is released so that the piston rod is relaxed and can be reset. For this principle to work, the force transmission from the actuator to the piston must be greater than the force transmission from the retaining bracket to the piston.

This mechanism allows the repeated injection and thus, a specific dose of the injection substance can be injected several times without the need to refill the syringe or to replace the syringe with another one, which saves time and reduces the risk of contamination.

The injection device of the present invention further comprises a needle guide which is adapted to allow guided injection at different injection sites. Preferably, the needle guide comprises a guide tube holder and a guide tube that is adapted to be removably couplable with an injection needle.

In a preferred embodiment and as shown in FIG. 9, the guide tube comprises or preferably consists of two parts, which, when assembled, form the complete guide tube. In this context, both counterparts are positively connected with each other. In particular, the guide tube in divided into two parts along its longitudinal axis and both parts-when assembled-form the guide tube. When both parts are connected to the guide tube, at least one channel is formed through which an injection needle can be guided. This channel lies preferably outside the axis of symmetry of the guide tube. In a preferred embodiment, two channels are formed when the two parts of the guide tube are assembled to the guide tube, preferably wherein one chancel is formed through which an injection needle can be guided, wherein this channel lies preferably outside the axis of symmetry of the guide tube, and one channel is formed through which a catheter can be guided, wherein this channel lies preferably in the axis of symmetry of the guide tube. Thus, at least one of the parts or each part comprise(s) respective protrusion(s) to form the channel(s) when assembled with the other part. In a preferred embodiment, one part of the guide type comprises a protrusion to form the catheter-channel when assembled with the other part to form the guide tube and the other part comprises a complementary protrusion to form the catheter-channel and comprises a further protrusion to form the needle-channel when connected to the other part to form the guide tube. Preferably, the catheter channel has such a dimension that the catheter tube just fits through which ensures that the guide tube itself is narrow, i.e., has a small diameter, and thus convenient for the patient. In a preferred embodiment, the catheter-channel has a diameter of about 1 mm to 6 mm, preferably of about 2 mm to 5 mm, more preferably of about 3 mm to 4 mm, and most preferably of about 3.6 mm. Additionally or alternatively, the guide tube at its widest point has a diameter of about 4 mm to 9 mm, preferably of about 5 mm to 8 mm, more preferably of about 6 mm to 7 mm, and most preferably of about 6.7 mm. Thus, in a most preferred embodiment, the catheter-channel has a diameter of 3.6 mm and the guide tube at its widest point has a diameter of 6.7 mm. This narrow construction allows the catheter tube to fit through the channel but not the balloon which is attached to the tip of the catheter (transurethral bladder catheter).

Accordingly, the two-part design/construction of the guide tube is advantageous since this allows insertion of the catheter into the guide tube. In particular, the catheter, i.e., the catheter tube is inserted into the protrusion of one part of the guide tube, and then, the other part of the guide tube which comprises the complementary protrusion is connected with the part of the guide tube which already holds the catheter. The part of the catheter comprising the balloon is outside the guide tube and protrudes out of the guide tube in the direction of the patient. If the guide tube was made from one piece, the catheter-balloon would have to be pushed through the channel of the guide tube, which is not possible since the channel is too narrow.

Furthermore, the construction of the guide tube comprising two channels, one for the needle and one for the catheter, ensures that the needle and the catheter tube run in parallel which prevents accidental puncturing of the catheter by the needle, which could occur if the injection device, in particular the guide tube, would not comprise a means for inserting the catheter.

In one embodiment, the needle guide is moveable for adjusting and changing the injection positions and adjusting and changing the position of the injection needle, respectively.

In particular, in one embodiment, the guide tube is rotatable about its longitudinal axis within the guide tube holder, wherein, the guide tube and the guide tube holder are toleranced to each other by means of a clearance fit.

In one embodiment, the injection device of the present invention is adapted to guide the injection needle to at least two injection positions, the location of which preferably differ from each other. This is preferably realized by a rotatable guide tube which comprises a channel which lies outside the axis of symmetry of the guide tube, and wherein the guide tube is adapted to guide the injection needle through said channel.

Injection at a plurality of injection sites, in particular along a crescent-shaped tissue requires a plurality of injection steps, in which a new injection site is alternately set and the injectable substance is deposited at the set injection site. This is important to ensure an equal distribution of the injection substance along the tissue leading to a successful tissue regeneration.

In one embodiment, one section of the guide tube comprises circumferential teeth directed outwards with respect to the surface of the guide tube, wherein the teeth together with a locking disk, which is arranged on the guide tube holder, ensures that the teeth of the guide tube engage with the locking disk when the guide tube will be rotated by a certain distance. Thus, the guide tube can have different locking positions in relation to the guide tube holder, wherein the distance between two locking positions, in particular two teeth of the guide tube, is preferably 15°. Thus, when moving, in particular rotating the guide tube, the position of the needle is changed by a certain distance, preferably by 15°. For ease of handling, the guide tube is in one embodiment operatively coupled to a handle, i.e. the handle is operatively coupled to the guide tube, which can be used to move, in particular to rotate the guide tube.

This feature is advantageous since it allows an equal distribution of the injection substrate along the tissue into which the substrate is to be injected and the correct position has not to be adjusted manually but it is predetermined by the interplay between the teeth of the guide tube and the locking disk of the guide tube holder. Accordingly, no unwanted variations in the injection positions occur.

As mentioned above, in one embodiment, the injection device of the present invention is further adapted to guide a catheter into the organ of interest, preferably into the urinary bladder. This is realized by the guide tube which further comprises in one embodiment a channel which lies in the axis of symmetry of the guide tube, and wherein the guide tube is adapted to guide the catheter through said channel. The catheter is used to empty the bladder and to fill it with isotonic fluid, like sodium chloride solution so that the bladder is filled and thus fixed, which facilitates targeted injection into the sphincter muscle.

In one embodiment, the injection device of the present invention comprises a cooling tool, which is preferably inserted into the holding unit and which is removable. In particular, the cooling tool is adapted to hold the syringe. The presence of the cooling tool has the advantageous effect that the injection substance is kept cool before administration to the subject which increases for example the shelf life of the injection substance. For example, if the injection tool is used for the administration of cell suspensions mixed with a collagen solution, which is the preferred use of the injection tool of the present invention, the cooling device is of highest importance since without cooling, the collagen solution would form a gel, which would make the suspension no longer injectable or if, only with sever loss in cell viability. Furthermore, if the injection substance comprises cells, for example muscle precursor cells, cooling of the cells ensures a high survival rate. In case the injection device of the present invention comprises a removable cooling tool, the cooling tool can be used for the transportation of the injection substance and the syringe comprising the injection substance, respectively, to the injection device.

In one embodiment, as shown in FIGS. 2 and 4, the cooling tool of the injection device of the present invention has facets, where the facets are preferably spaced at about 15° apart from each other and run around the cooling tool. Thus, each facet corresponds to 15°. The cooling unit is clamped with the holding unit in such a way that after a rotation of the cooling unit by 15°, the cooling tool snaps back into place with every 15° rotation, which allows the operator, for example the physician to let go of the device in any position without it changing its position. In one embodiment, the injection device of the present invention comprises the above-mentioned cooling tool which is rotatable, in particular about its longitudinal axis in order to allow mixing of the injection substance which is comprised in the syringe. This is particularly useful if the injection substance is not a homogenous substance respective solution, but a heterogeneous mixture, e.g., suspension which is composed of at least two immiscible substances, for example cells (as particles) and the surrounding (preferably fluid) medium, e.g., a carrier solution, like a collagen solution. The rotation of the cooling unit prevents that one phase, e.g., the cells, settle down and ensures that both phases in case that the substances are both fluids, or in case of a suspension, e.g., the cells and the medium are homogenously distributed within the syringe.

As mentioned above, the injection device of the present invention comprises in one embodiment a connection structure, which operatively connects at least the holding unit with the needle guide. The connection structure is preferably designed as elongated struts, especially elongated rods onto which the other parts of the injection device of the present invention are mounted either directly or with further connection means. Preferably, the holding unit is mounted directly onto the connection structure (and the actuating unit is mounted onto the holding unit), wherein the needle guide is mounted onto the connection structure via a further connection means, i.e., a positioning structure as defined below. Accordingly, the connection structure extends along the axial reference plane of the injection device (see FIG. 1).

The connection structure is preferably designed so that the needle guide is slidable along its rods and that the holding unit (including the actuating unit) is slidable along its rods. The needle guide and the holding unit can slide independently from each other.

Sliding of the holding unit including the actuating unit is performed to advance the injection needle, when connected to the syringe placed in the holding unit of the injection device and guided through the needle guide. During the injection procedure, the actuating unit including the holding unit is advanced in direction to the organism, i.e., in direction to the injection site, until the tip of the needle penetrates sufficiently far into the site of injection, preferably a human or animal tissue, more preferably into a sphincter muscle, even more preferably into the external urinary sphincter muscle, in particular preferred into human sphincter muscle or the external urinary sphincter muscle. Sliding of the actuating unit and the holding unit, respectively can either be performed manually or motor driven, for example artificial intelligence (AI) controlled as explained further below.

Sliding of the needle guide is advantageous to adjust the distance between the guide tube/the injection needle when guided through the guide tube and the head of the ultrasound probe, which can be connected to the injection device of the present invention as described below to adapt the injection device of the present invention to the anatomy of the subject to be treated. The connection structure is preferably further adapted to be removably coupleable with an imaging device, preferably an ultrasound probe. The ultrasound probe is advantageous for the observation of the tissue to which the injection substance is to be administered, in particular to determine the optimal position and injection depth of the injection needle. An ultrasound probe guided injection is usually more precise than injection guided by cystoscopy and thus, advantageous.

Accordingly, in one embodiment the connection structure further comprises an attachment means for the imaging device, in particular for the ultrasound probe.

Thus, in one embodiment, the injection device of the present invention comprises an imaging device, which is preferably an ultrasound probe, and which is removably coupled to the injection device of the present invention via the connection structure. Thus, the connection structure has in one embodiment attachment means for the imaging device.

The injection is normally performed by trained physicians in a hospital setting and precision of the injection can be further improved via artificial intelligence (AI)-enabled ultrasound interpretation, which allows for example to perform targeted, preferably motorized, needle insertion, and preferably automatically confirms successful puncture of the respective tissue, for example the external urethral sphincter. This enables the users to rapidly and precisely localize the correct injection site, and in particular injection depth and/or angle without the need for manual ultrasound interpretation and thus, lowering intersurgeon and intrasurgeon variability. The principle of AI-enabled ultrasound interpretation is for example described in Brattain et al., Biosensors 11 (2021), 522.

In one embodiment, the injection device of the present invention further comprises a positioning structure for the needle guide, i.e., the needle guide is removably and movably connected to respectively onto the positioning structure, wherein the positioning structure preferably comprises a rail, which is connected with the connection structure and extends in perpendicular direction to the connection structure, and more preferably further comprises a slide, which is removably and movably couplable to said rail.

The positioning structure allows the needle guide to move into different directions, which in turn allows the injection needle when inserted into the needle guide to move with the needle guide. Thus, in one embodiment, the guide tube holder of the injection device of the present invention is movable.

In particular, in one embodiment the needle guide including the guide tube and the guide tube holder is movable in relation to the holding unit, preferably towards the holding unit and away, i.e. the distance between the holding unit and the guide tube holder is adjustable, wherein the distance between the guide tube holder and the imaging device, in particular the shaft of the ultrasound probe, when connected to the connection structure of the imaging device, remains the same. In particular, the needle guide can be moved along the connection structure and thus, the distance between the needle guide and the imaging device, in particular the shaft of the ultrasound probe, remains the same. This allows to adjust the distance between the guide tube (and the injection needle when inserted) and the head of the ultrasound probe. In one embodiment, this is realized by the rail to which the guide tube holder including the guide tube is connected, wherein the rail is movable in the above-indicated direction.

In one embodiment, the needle guide including the guide tube holder and the guide tube is movable in relation to the imaging device, in particular in relation to the shaft of the ultrasound probe, and preferably towards the imaging device and away, i.e., the distance between the needle guide and the imaging device is adjustable. In one embodiment, this is realized by the slide which is connected to the rail, wherein the slide to which the guide tube holder including the guide tube is connected can slide along the rail.

In one embodiment, the needle guide including the guide tube and the guide tube holder is pivotable about a pivot axis. This is also realized by the slide which is connected to the rail via a pivot bearing.

Due to the differences in the anatomy of the organisms to be treated, for example the anatomy of the vagina and the bladder of female patients and thus, of the position of external urethral sphincter muscle, it is important that the distance between the guide tube, and thus the needle when inserted into the guide tube, and the ultrasound probe can be adjusted. For the same reason, it is advantageous that the distance between the guide tube, and thus the needle when inserted into the guide tube, and the head of the ultrasound probe can be adjusted.

Furthermore, the characteristic that the guide tube holder including the guide tube is pivotable is advantageous since this allows the adaption of the injection angle. The injection angle (angle of penetration, angle of inclination) is the angle between the longitudinal direction of a tissue, in particular of a muscle, e.g., the sphincter muscle into which an injection substance is to be introduced, and the injection needle.

In a further embodiment, the present invention relates to the injection device of the present invention and an injection needle, wherein at least the front part of the injection needle which protrudes from the guide tube towards the injection site is bend. In other words, the present invention relates to the injection device of the present invention which further comprises an injection needle, wherein the injection needle which protrudes from the guide tube towards the injection site is bend. Preferably, the injection needle per se is not bend, but the curvature of the needle is caused by a grinding in the guide tube, which results in that the injection needle exits the guide tube in a curved shape.

In particular, the needle has a curvature that is directed radially outwards relative to the axial orientation of the ultrasonic probe and the guide tube, respectively. In a preferred embodiment, the injection needle is bent by up to 10°, preferably by less than 10°, more preferably by less than 7.5°. In a further preferred embodiment, the injection needle is bent by, i.e., its curvature is 3° to 10°, preferably 3° to 9°, more preferably 3° to 8°, more preferably 3° to 7.5°, more preferably 3° to 7°, more preferably 3° to 6.5° or 4° to 6.5°, more preferably 3° to 6° or 4° to 6.5°, more preferably 3° to 5.5°, or 4° to 5.5°, more preferably 3° to 5°. Most preferably, the injection needle is bent by, i.e., its curvature is between 5° and 7.5°, preferably 5° or 7.5°. In a preferred embodiment, the front part of the needle per se which protrudes the guide tube has a curvature of 3° to 6°, preferably of 4° to 5°, most preferably 5°, and the guide tube can be arranged to adjust the curvature of the needle, preferably by further 1° to 3°, preferably less than 2.5°, i.e. the guide tube can be adjusted upwards or downwards in relation to its longitudinal axis.

The curvature of the needle is advantageous in order to avoid the needle piercing through the tissue into which the injection substance is to be injected, but instead the injection angle of the needle is shifted by a certain curvature radially outward or inward with respect to the center of the longitudinally extending tissue, so that the direction of injection is shifted radially in the longitudinal direction of the tissue.

In a preferred embodiment, the injection needle is attached to the syringe, in particular to the nozzle of the syringe. The connection between the nozzle of the syringe and the needle is leak-free. In one embodiment, the leak-free connection is realized with the Luer taper. In one embodiment the needle is connected via an intermittent tube to the nozzle of the syringe. In a preferred embodiment the connection of one end of the intermittent tube to the nozzle and of the other end of the tube to the needle is realized with the Luer taper.

In one embodiment, the injection device of the present invention is connected to a mount, for example a bed or floor mount, preferably via a plinth which is part of the connection structure, to ensure a stable position of the injection device in relation to the organism to be treated.

In a further embodiment, the present invention relates to a medical device which is adapted for injection of an injection substance into an organism. The medical device of the present invention comprises the injection device of the present invention and a syringe with a syringe body and a plunger including a piston as well as an injection needle, wherein the syringe is arranged in the holding unit of the injection device, and/or wherein the injection needle is arranged in the needle guide. The present invention further relates in one embodiment to the use of the syringe comprising the injectable substance and the injection needle, respectively in the injection device of the present invention and the medical device of the present invention

In one embodiment, the medical device further comprises an imaging device, preferably an ultrasound probe.

In one embodiment, the injection device of the present invention and the medical device of the present invention are adapted to be removably couplable with a bed or floor mount, for example a brachytherapy stepper arm.

The present invention further relates to a method for injecting an injection substance into an organism, wherein the injection device and the medical device, respectively, of the present invention is used comprising at least a step of a dosed injection of the injection substance into the organism by displacement of the lever of the actuating unit of the medical device from a first position to a second position as described in detail above.

Furthermore, the present invention relates to a method for the treatment of muscle dysfunctions in a subject, for example a smooth muscle or skeletal muscle dysfunction, preferably a skeletal muscle dysfunction. In a preferred embodiment, the skeletal muscle dysfunction can be for example a dysfunction of a sphincter. Sphincters are circular muscles that serve as valves to open and close certain parts of the body. There are for example six different sphincters within the digestive system, the upper esophageal sphincter, lower esophageal sphincter, pyloric sphincter, sphincter of Oddi, ileocecal sphincter, and anal sphincter. Further sphincter muscles are present in the body, for example the urethral sphincter. The action of sphincters may happen involuntarily through the autonomic nervous system or maybe under some voluntary control through the somatic nervous system. If a sphincter loses muscle tone or has too much tone (spasticity), symptoms and illness can follow. This can include urinary retention, in which the bladder cannot empty completely. Sphincter issues can also cause bladder and fecal incontinence, or the inability to control the bladder or bowels.

Thus, the injection device/medical device of the present invention can be used in a method for the treatment of a disease related to the above-mentioned sphincter muscles, preferably of a disease related to a defect or dysfunction of the urethral sphincter, more preferably of the external urethral sphincter muscle.

Thus, in a further preferred embodiment, the skeletal muscle dysfunction is a defect of a sphincter, preferably of the external urethral sphincter muscle. Accordingly, the present invention relates in one embodiment to a method for the treatment of diseases which are related to muscle dysfunction, which include but are not limited to (female) urinary incontinence, male urinary incontinence for example after prostatectomy, and anal incontinence, but preferably to the treatment of stress urinary incontinence.

Urinary incontinence, the involuntary loss of urine, is a major medical issue with approximately half of the female population affected over 45 years and 17% of men over 70 years of age. Continence and micturition involve a balance between urethral closure and detrusor muscle activity. There are different types of urinary incontinence, such as stress and urge incontinence. Stress urinary incontinence (SUI) is the loss of small amounts of urine associated with coughing, laughing, sneezing, exercising or other movements that increase intra-abdominal pressure and thus increase pressure on the bladder. The external striated urethral sphincter, which is made of skeletal muscle and therefore is under voluntary control of the somatic nervous system is, for the most part, responsible for preventing SUI. Damage to the external urethral sphincter occurs mainly during childbirth, surgical treatments or as an effect of aging. SUI is a disease affecting over 200 million people worldwide and is twice as common in women as in men, decreasing the quality of life of patients due to limited daily activities, unpleasant sensation, odor and infections caused by wet diapers. The incurring healthcare costs are significant.

Treatment options of SUI include mainly non-surgical therapy (bladder training, dietary modifications), drug therapy and surgical therapy. These therapies offer only short term relief and their overall success is often limited by complications (invasiveness of v surgery, damage to surrounding tissues, leading to increased urinary infection rates) or side-effects (drugs, tissue damage by non-degradable biomaterials, etc.). However, great advances in cell therapy approaches to treat urinary incontinence have been made which can restore the sphincter function in patients with SUI; see for example WO 2019/215090 A1. In general, the injection device/medical device of the present invention can be used for injecting any substance into a tissue, but it is preferably used for injecting cells which are able to regenerate the tissue, for example the sphincter muscle tissue.

In a preferred embodiment, the method of the present invention comprises the injection of the injection substance, which comprises at least muscle-derived precursor cells and optionally further ingredients, e.g., a carrier solution for the cells, for example a collagen solution, into the respective tissue, in particular into muscle tissue and preferably into muscle tissue of sphincter, preferably the (external) urethral sphincter muscle or the (external) anal sphincter muscle with the medical device of the present invention.

In one embodiment, the method of the present invention, which is outlined in more detail below, further comprises neuro-muscular electromagnetic stimulation (NMES) of the pelvic floor of the patient as described in WO 2019/215090 A1. NMES-treatment following the injection of the cell suspension supports muscle and nerve regeneration by activating muscle-nerve cross-talk and induces the maturation of neuromuscular junctions.

As mentioned above, as injectable substance preferably muscle-derived precursor cells are used in the method of the present invention, which are optionally mixed with a carrier solution, for example collagen solution as described in WO 2019/215090 A1. Furthermore, respective treatment approaches are described in WO 2019/215090 A1, US2009/0098094 A1, WO 2004/096245 A2, and WO 2008/104883 A1, which are all incorporated herein by reference. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

For the avoidance of any doubt it is emphasized that the expressions “in some embodiments”, “in a certain embodiments”, “in certain instances”, “in some instances”, “in a further embodiment”, “in one embodiment” and the like are used and meant such that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices.

### DESCRIPTION OF THE FIGURES

FIG. 1: Schematic view of the injective device according to one embodiment of the present invention including dimensions (in mm and indicated with arrows).

FIG. 2: Perspective view of the injective device according to one embodiment of the present invention.

FIG. 3: Schematic view of the injective device according to one embodiment of the present invention including an imaging device, here an ultrasound probe including dimensions (in mm and indicated with arrows).

FIG. 4: Perspective view of the injective device according to one embodiment of the present invention including an imaging device, here an ultrasound probe.

FIG. 5: Enlarged and detailed view of one embodiment of the actuating unit of the injection device of the present invention showing the lever in the first position (A) and in the second position (B).

FIG. 6: Enlarged and detailed view of the syringe and the injection needle when placed within the cooling tool of the injection device of the present invention.

FIG. 7: Enlarged and detailed view of one embodiment of the guide tube and of the big part of said guide tube (comprising three quarters of the guide tube), respectively, of the injection device of the present invention including dimensions (in mm and indicated with arrows) and the specification of radii (R in °). A) complete guide tube; B) big part of the guide tube; C) cross section of the big part of the guide tube; D) cross section of the disk with radially projected teeth of the big part of the guide tube.

FIG. 8: Enlarged and detailed view of the small part of one embodiment of the guide tube (comprising one quarter of the guide tube) of the injection device of the present invention including dimensions (in mm and indicated with arrows) and the specification of radii (R in °). A) small part of the guide tube; B) cross section of the small part of the guide tube; C) cross section of the disk with radially projected teeth of the small part of the guide tube; D) radial section of the small part of the guide tube.

FIG. 9: Schematic view of the assembly of the guide tube, i.e., assembly of the big part and the small part of the guide tube to the complete guide tube.

FIG. 10: Schematic view of one embodiment of the locking disk of the injection device of the present invention including dimensions (in mm and indicated with arrows) and the specification of radii (R in °).

FIG. 11: Schematic view of one embodiment of the positioning structure (comprising rail and slide) including the guide tube holder and the locking disk as well as a cross section of the guide tube within in the guide tube holder of the injection device of the present invention and a corresponding enlarged view of the locking disk and the cross section of the guide tube.

FIG. 12: Schematic view of one embodiment of the rail of the injection device of the present invention including dimensions (in mm and indicated with arrows) and the specification of radii (R in °). A) side view of the rail; B) front view of the rail.

FIG. 13: Schematic view of one embodiment of the slide of the injection device of the present invention including dimensions (in mm and indicated with arrows) and the specification of radii (R in °).

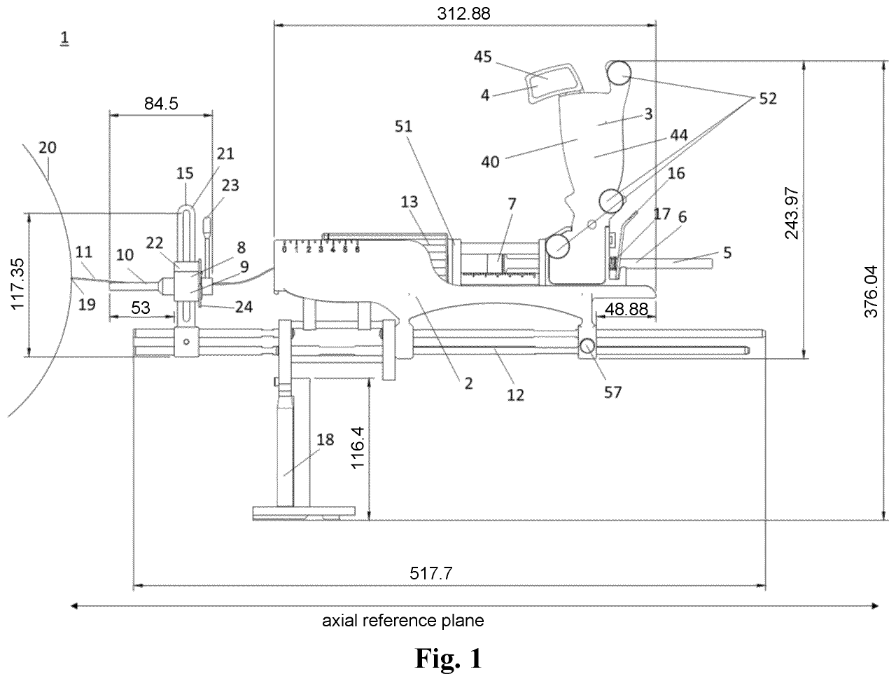

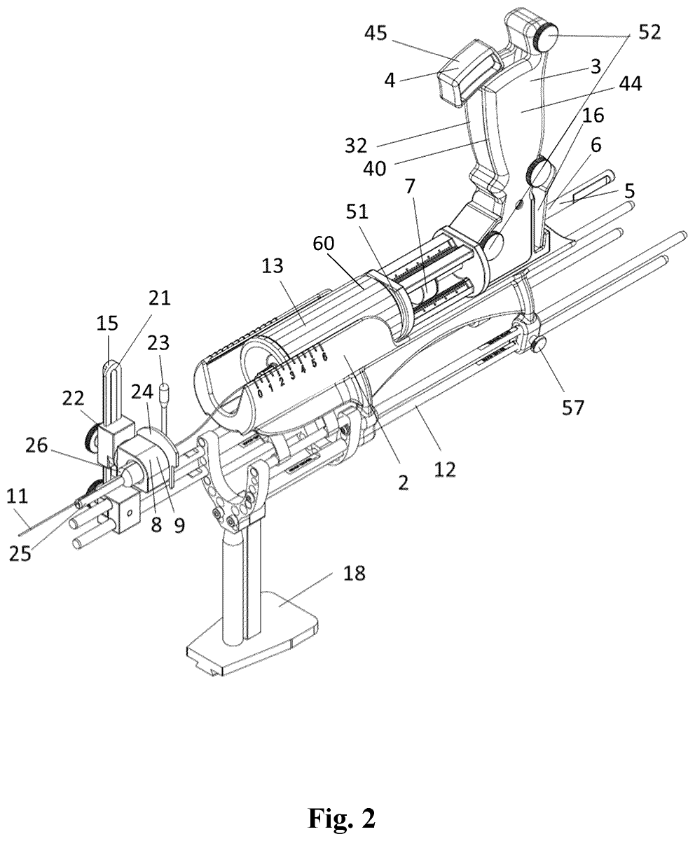

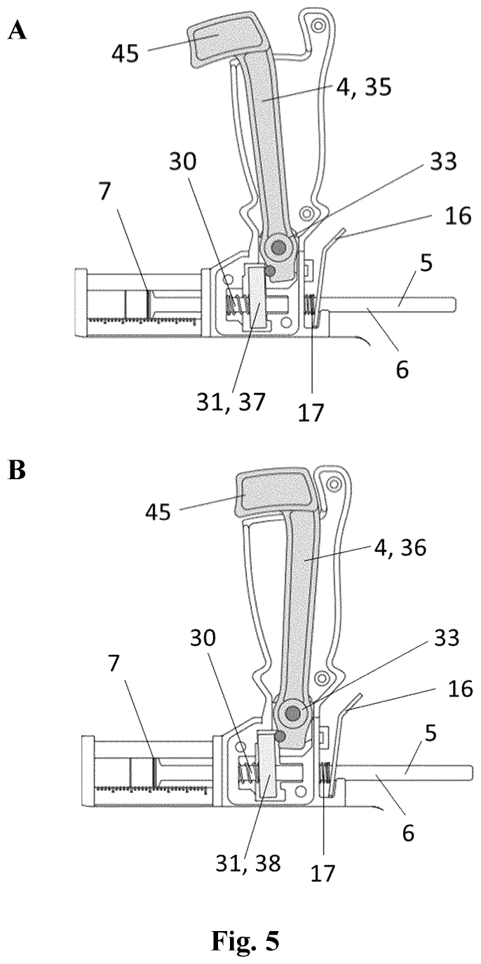

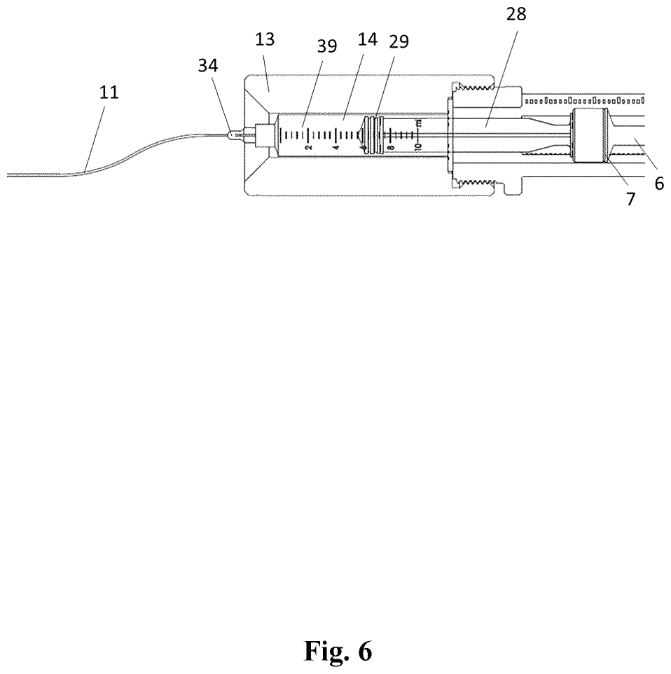

One embodiment of the injection device 1 of the present invention is shown in FIG. 1 and FIG. 2, wherein the location of the syringe 14 within the injection device is shown in FIG. 6, and details about the actuating unit 3, in particular the different positions of the lever, are shown in FIG. 5. The injection device 1 of the present invention comprises at least a holding unit 2 for the syringe 14, i.e., it is designed to hold the syringe 14, an actuating unit 3, a needle guide 8, and a structure which connects at least the holding unit 2 with the needle guide 8, i.e., the connection structure 12.

In particular, the injection device 1 of the present invention comprises
a holding unit 2, which is adapted to hold the syringe 14;
an actuating unit 3, which is removably coupled to the holding 2 unit and which is couplable with the syringe 14, in particular with the plunger 28 of the syringe 14, when the holding unit 2 holds the syringe 14, wherein the actuating unit 3 comprises a lever 4 and a piston 5, wherein a displacement of the lever 4 from a first position 35 to a second position 36 results in a movement, in particular an advancing movement of the piston 5 along its longitudinal axis, in particular in a forward direction, wherein the actuating unit 3 is adapted to transfer the advancing movement at least partly to the syringe 14, in particular to the plunger 28 of the syringe, when the syringe 14 is coupled with the actuating unit 3;
a needle guide 8, which is adapted to be removably couplable with an injection needle 11 and which is adapted to changing the injection position of the injection needle 11, wherein the needle guide 8 preferably comprises a guide tube holder 9 and a guide tube 10, wherein the guide tube 10 is movable with respect to the guide tube holder 9, in particular rotatable within in the guide tube holder 9, for changing the injection position of the injection needle 11; and
a connection structure 12 which connects the holding unit 2 and the needle guide 8.

In particular, the holding unit 2, the actuating unit 3 and the needle guide 8 are arranged in series in such a way that they are operatively connected, when the injection device is used, i.e., when a syringe 14 is inserted into the holding unit and when an injection needle 11 is attached the nozzle 34 of the syringe 14 and guided through the guide tube 10, wherein the connection structure 12 connects the different elements. In particular, the connection structure 12 connects the holding unit 2 with the needle guide 8 and thus, also indirectly connects the actuating unit 3 with both mentioned components of the injection device 1, since the actuating unit 3 is mounted onto the holding unit 2.

FIG. 1 and FIG. 2 show one embodiment of the injection device 1 of the present invention and FIG. 1 further shows the orientation of the device relative to an organism, in particular relative to the injection site 19 of the organism 20. More particularly, FIG. 1 shows the orientation of the injection device 1 relative to the external urethral sphincter muscle (schematic, not drawn to scale). The length of the external urethral sphincter muscle is between about 10 mm and 18 mm and the thickness is between about 1.5 mm to 5 mm (Morgan et al., J Urol. 182 (2009), 203-209). FIG. 6 shows the orientation of the syringe 14 within the injection device 1 of the present invention, in particular within the holding unit 2 of the injection device 1 of the present invention.

The injection device 1 of the present invention comprises a holding unit 2 for the syringe 14, and thus, the syringe 14 can be inserted into the holding unit 2. The holding unit 2 can be made of any material that allows the functionality of the holding unit and is preferably suitable for medical purposes, and can be designed as disposable and/or can be made for example of a plastic material, or steel, preferably stainless steel. One example of the preferred plastic material is a polyamide, preferably polyamide-12, and most preferably Fine polyamide PA 2200 for EOSINT P. Same regards to the other parts of the injection device 1 of the present invention, if not specified otherwise. Preferably, any plastic material can be used as long as it is certified for medical use, and can be cleaned and/or sterilized.

In principle, the holding unit 2 can have any design as long as it holds the syringe body 39 in place, wherein the plunger of the syringe 28 is movable. In particular, the plunger of the syringe 28 can make an advance movement along its longitudinal axis in direction to the outlet/the tip of the syringe 14 and can make a backward motion along its longitudinal axis in the opposite direction, i.e., in direction of the actuating unit 3 of the injection device 1.

In particular, the injection device 1 of the present invention comprises the holding unit 2 which holds the syringe body 39 in place so that it does not change its position within the holding unit 2. In one embodiment, the holding unit 2 and the syringe 14, in particular of the syringe body 39 are positively connected to each other. In another embodiment, the syringe body 39 and the holding unit 2 are toleranced to each other by means of a clearance fit so that the syringe body 39 can rotate within the holding unit 2 along its longitudinal axis.

As mentioned above, the injection device 1 of the present invention is preferably designed for repeated dosed injections at a specific predetermined amount. In this context, the holding unit 2 of the injection device 1 of the present invention comprises in one embodiment number markers based on which the operator can estimate how many injections have already been performed. In particular, this can be estimated based on the position of the piston of the syringe 29.

In one embodiment, the holding unit 2 of the injection device 1 of the present invention further holds a cooling tool 13, which is preferably a cooling tool for the syringe 14 and its content inside the syringe body 39, respectively. In one embodiment, the cooling tool 13 of the injection device 1 of the present invention is removable, i.e., it can be removed from the holding unit 2, for example for transportation. In one embodiment, the cooling tool 13 is removably fixed to the holding unit 2 so that it does not change its position within the holding unit 2 after it has been inserted into the holding unit 2 and it is also not rotatable, i.e., the holding unit 2 and the cooling tool 13 are positively connected to each other.

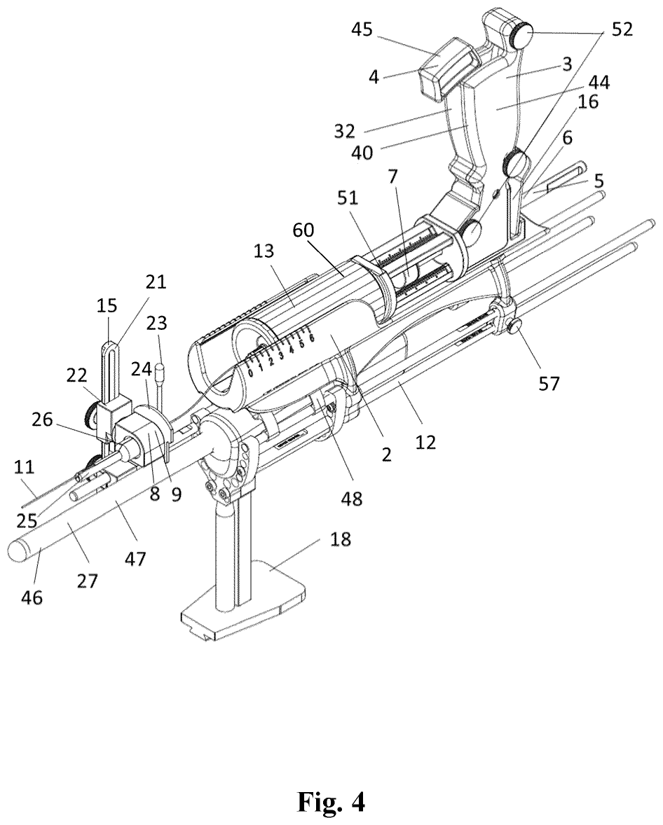

In a preferred embodiment, the cooling tool 13 is removably fixed to the holding unit 2 so that it does not change its position within the holding unit 2 after it has been inserted into the holding unit 2, but it is rotatable along its longitudinal axis. In the latter case the cooling tool 13 and the holding unit 2 are tolerance to each other by means of a clearance fit so that the cooling tool 13 can rotate within the holding unit 2 along its longitudinal axis. In a further preferred embodiment, the cooling tool 13 has facets 60 as shown in FIGS. 2 and 4, wherein the facets 60 are preferably spaced apart from other and run around the cooling tool 13. The facets are preferably evenly distributed around the cooling tool, i.e., have the same distance from each other. Preferably, the facets are spaced at about 5° to 25° apart from each other, more preferably at about 10° to 20°, more preferably at about 12° to 18°, more preferably at about 14°to 16°, and are most preferably spaced apart at about 15° from each other. Accordingly, the cooling unit is clamped with the holding unit in such a way that after a rotation of the cooling unit by the above indicated degree, preferably by 15°, the cooling tool snaps back into place with every 15° rotation.

In principle, the cooling tool can have any design as long as a syringe 14 can be inserted, i.e., the cooling tool 13 has an opening for insertion of the syringe 14 and/or as long as it holds the syringe 14 in place. In a preferred embodiment, the cooling tool is a hollow cylinder which surrounds the syringe, in particular the syringe body 39, which preferably contains the content to be cooled, from all sides. The cooling tool 13 can be made of aluminum or a ferrous alloy such as stainless steel and is cooled on ice before use, i.e., before the cooling tool 13 including the syringe 14 including the substance to be injected is connected to the holding unit 2 of the injection device 1 of the present invention. Preferably, the cooling tool 13 is made of any material with optimal isolation properties. In particular, the cooling tool 13 comprises in one embodiment a screw thread 51 with which it can be screwed onto the actuating unit 3 of the injection device 1 of the present invention, wherein the actuating unit 3 including the cooling tool 13 including the syringe 14 and the substance to be injected, is connected with the holding unit 2 of the injection device 1 of the present invention as shown for example in FIGS. 1 and 2.

As mentioned above, the cooling tool 13 is designed to hold the syringe 14. In particular, it holds the syringe body 39 in place, wherein the plunger of the syringe 28 is movable. In particular, the plunger of the syringe 28 can make an advance movement along its longitudinal axis in direction to the outlet/the tip of the syringe 14 and can make a backward motion along its longitudinal axis in the opposite direction.

In particular, the injection device 1 of the present invention comprises the holding unit 2 which holds the cooling tool 13 which holds the syringe body 39 in place so that it does not change its position within the cooling tool 13. In one embodiment, the syringe body 39 and the cooling tool 13 are toleranced to each other by means of a clearance fit so that the syringe body 39 can rotate within the cooling tool 13 along its longitudinal axis and in another embodiment, the syringe body 39 and the cooling tool 13 are positively connected to each other.

In a preferred embodiment, the syringe body 39 is fixed within the cooling tool 13 so that it does not change its position within the cooling tool and so that it is also not rotatable, i.e., the syringe body 39 and the cooling tool 13 are positively connected to each other. Accordingly, in one embodiment, neither the cooling tool 13 nor the syringe body 39 can change its position in relation to the holding unit 2. In a preferred embodiment, the cooling tool 13 is rotatable as explained above and thus, the syringe 14, in particular the syringe body 39 is rotatable within the holding unit 2, since the syringe 14, in particular the syringe body 39 is removably fixed within the cooling tool 13.

The syringe 14 can have any size as long as it fits into the holding unit 2 and the cooling tool 13, respectively. In one embodiment, the syringe 14 holds at least 10 μl, at least 400 μl, at least 600 μl or at least 800 μl volume, preferably at least 400 μl or 600 μl. In a preferred embodiment, the injection syringe 14 holds at least 1 ml, at least 4 ml, at least 6 ml, or at least 8 ml volume, preferably at least 4 ml (for 10×400 μl injections (low dose)), or 6 ml (for 15×400 μl injections (high dose)). Most preferably, a 10 ml syringe 14 is used in accordance with the present invention, for example the syringe Braun Omnifix 10 ml luer-lock.

The injection device 1 of the present invention further comprises an actuating unit 3. One embodiment of the actuating unit 3 is shown in more detail in FIG. 5. The actuating unit 3 can be made of any material that allows handling of the actuating unit and is preferably suitable for medical purposes and can be designed as disposable and/or can be made for example of a plastic material, or steel, preferably stainless steel. One example of the preferred plastic material is a polyamide, preferably polyamide-12, and most preferably Fine polyamide PA 2200 for EOSINT P.

In one embodiment, the actuating unit 3 is mounted onto the holding unit 2 of the injection device 1 of the present invention, i.e., it is slid onto the holding unit 2 when the injection device 1 of the present invention is assembled. In particular, the actuating unit 3 is removably coupled to the holding unit 2 and is couplable with the syringe 14 when the holding unit 2 holds the syringe 14. In one embodiment, the actuating unit 3 is removably coupled to the holding unit 2, removably coupled to the cooling unit 13, and is couplable with the syringe 14 when the holding unit 2 holds the syringe 14.

In principle, the actuating unit 3 can have any design as long as it allows dosed injection of an injectable substance. In particular, the actuating unit 3 can have any design as long as it allows dosed injection of an injectable substance, which is present in the syringe 14. In one embodiment, the actuating unit 3 is designed as shown in FIG. 5.

Thus, in a preferred embodiment, dosed injection is realized by an actuating unit 3 which, when actuated, for example by displacing the lever 4 of the actuating unit 3 from a first position 35 as shown in FIG. 5A to a second position 36 as shown in FIG. 5B, preferably pivoting the lever 4 from the first position 35 to the second position 36 about a pivot axis, ensures that the piston 5 of the actuating unit is displaced, i.e., advanced, along the longitudinal axis by a certain displacement distance, i.e. from a first position to a second position, in the direction of the piston stop (forward direction), wherein the piston 5 when advanced exerts force onto the plunger of the syringe 28 so that it is also advanced along its longitudinal axis by a certain distance in direction to the outlet of the syringe 14 so that the injection substance present in said syringe is released, i.e., injected into the desired injection site 19. In a preferred embodiment, the displacement distance of the lever 4 and the piston 5, respectively, is adapted in that 400 μl of injection substrate is dispensed from the syringe 14 via the nozzle 34 as described above per piston stroke, i.e., each trigger of the lever results in that 400 μl of injection substrate is dispensed from the syringe 14. The distance which is covered depends on the dimension of the syringe, but when a 10 ml syringe is used, for example the syringe Braun Omnifix 10 ml luer-lock with a 15.9 inner diameter, the displacement distance of the lever is preferably 20 mm, and/or the displacement distance of the piston is preferably 2 mm.

In one embodiment, the actuating unit 3 of the injection device 1 of the present invention further comprises a lever shell 32. In one embodiment, the actuating unit 3 of the injection device 1 of the present invention further comprises a cover of the lever shell 44, which is preferably connected with the lever shell 32 via knurled screws 52. The lever shell 32 and the cover of the lever shell 44 together form the housing 40 of the lever. In particular, at least parts of the lever 4 and the actuator 31 are placed inside the housing 40 and wherein the housing 40 has at least a recess to access the lever 4, i.e., to operate the lever 4. In one embodiment, the lever actuation point 45, i.e. the handle of the lever 45, is outside of the housing 40 of the actuating unit 3.

The lever shell 32 including its cover 44 can be used as a handle which facilities the handling and operating of the injection device 1 of the present invention.

The transmission of the force from the lever 4 to the piston 5 occurs preferably via an actuator 31, like a clamping piece. Accordingly, the lever 4, the actuator 31 and the piston 5 are operatively connected. In particular, the lever 4 and the actuator 31 are operatively connected, for example by one or more bolts 33. When the lever 4 is actuated from a first position 35 to a second position 36, the actuator 31 is tilted, i.e. moved from its first position 37 to its second position 38, so that it clamps the piston rod 6 and advances the piston 5 along its longitudinal axis as described before. In other words, displacement of the lever 4 from the first position 35 to the second position 36 causes the lever 4 to engage the actuator 31 via a pivot point in such a way that the actuator 31 is tilted which results in a frictional connection between the actuator 31 and the piston rod 6 by causing them to frictionally engage with each other, which then leads to the aforementioned advance movement. The frictional connection between the actuator 31 and the piston 5 of the actuating unit 3 can be caused by (slightly) tilting, in particular pivoting the actuator 31 with respect to a perpendicular orientation of the actuator 31 and the piston 5.

The displacement of the piston 5 of the actuating unit 3 is completed, i.e. the second position is reached, when the actuator 31 abuts against the stop in the housing 40 of the actuating unit 3, in particular against a compression spring 30, which is placed at the level of the actuator 31 at the inner wall of the housing 40, in particular the inner wall of the part of the housing 40 which faces the syringe 14 when inserted into the injection device and the cooling tool 13, respectively and/or the lever 4, i.e., the lever handle 45 abuts against the stop 59 of the housing 40.

In one embodiment, the actuating unit 3 of the injection device 1 of the present invention further comprises a retaining element 16, preferably a retaining bracket, also called clamping plate, which embraces the piston rod 6 of the actuating unit 3 at its distal end, i.e., the end opposite of the piston stop 7. The retaining element is hold in a recess exterior of the housing 40 of the actuating unit 3 and is tensioned by means of a compression spring 17. The retaining element 16 frictionally engages the piston rod 6 in a retaining position in such a way that the piston 5 is held in position and prevents the piston 5 from being retracted along its longitudinal axis, i.e. moved backwards (opposite direction to the above-mentioned forward direction) when the lever 4 and the actuator 31, respectively, move from the second position to the first position. This allows the re-actuation of the actuating unit 3, i.e. the movement of the lever 4 again from a first position 35 to a second position 36, resulting in renewed metered substrate delivery.

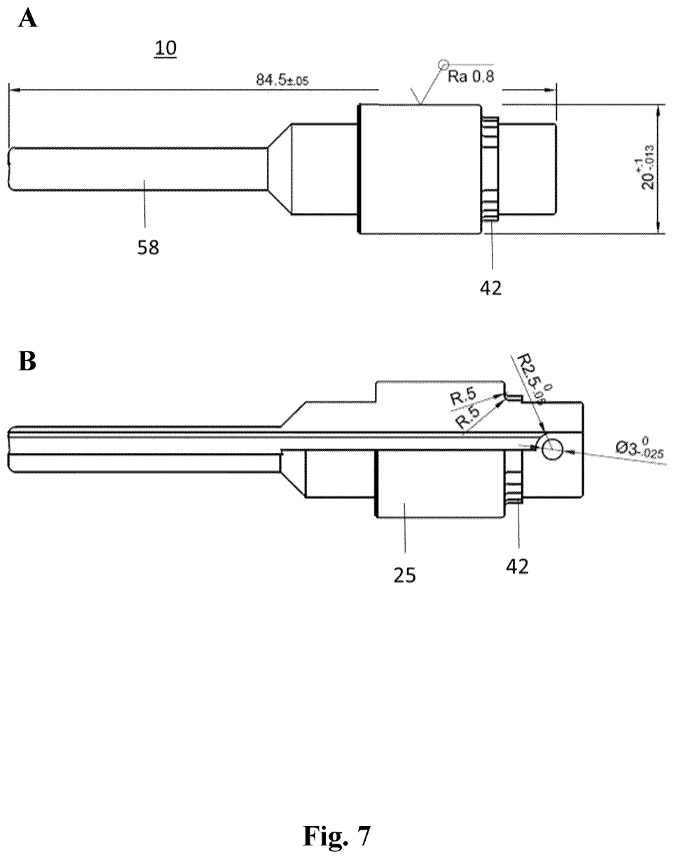

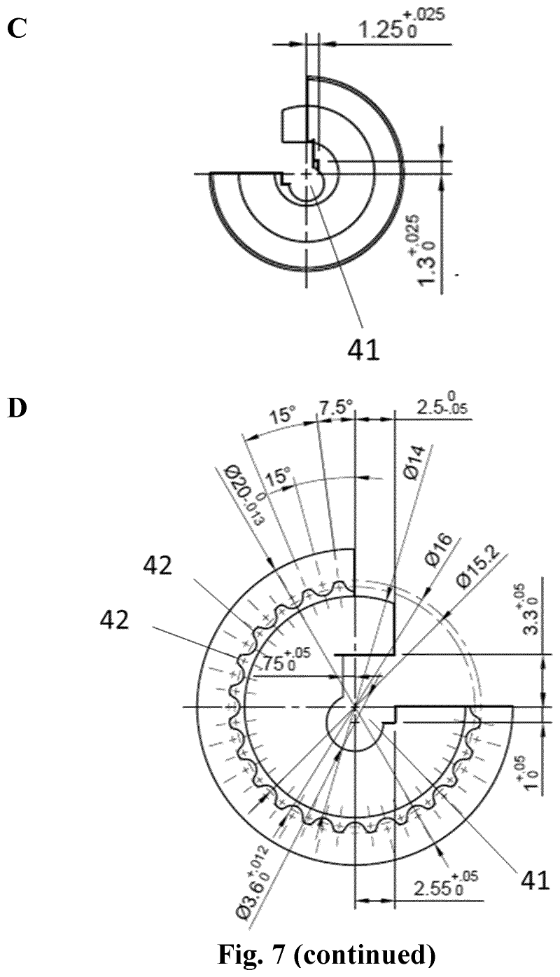

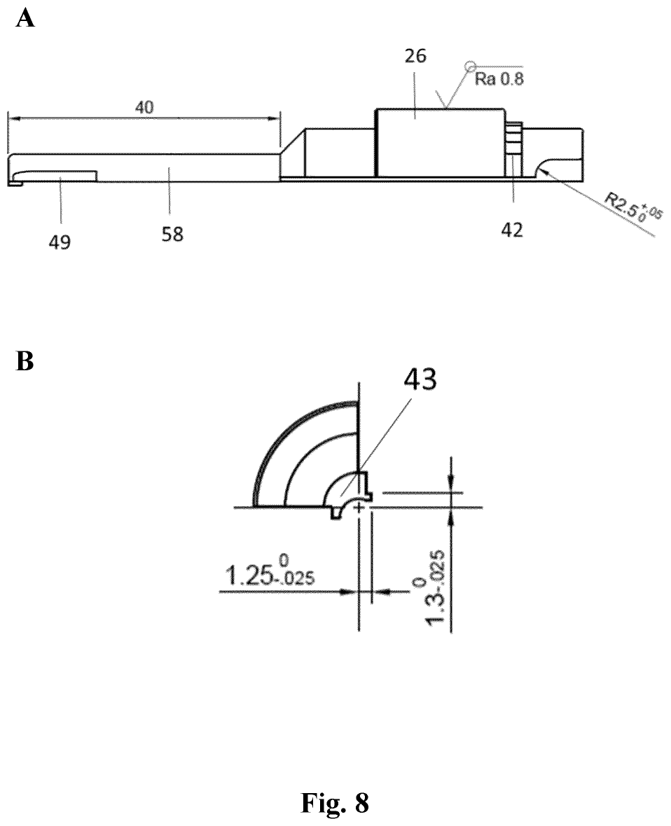

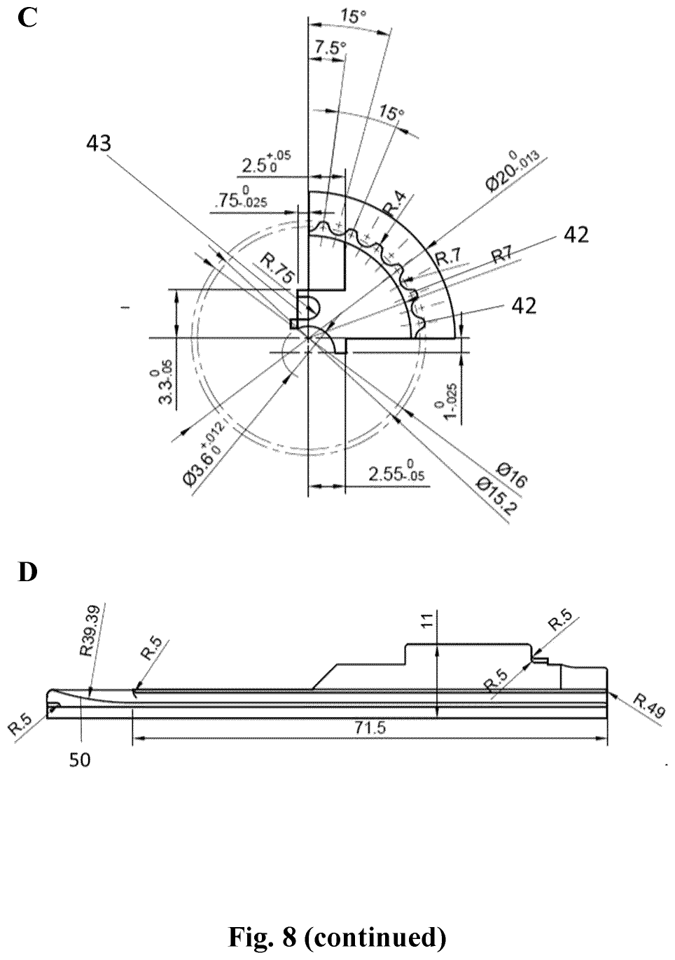

The injection device 1 according to the present invention further comprises a needle guide 8, wherein the needle guide 8 comprises at least a guide tube 10 and a guide tube holder 9 and is in particular adapted in such a way that an injection needle 11, when coupled to the injection device 1 of the present invention, and in particular to an syringe 14 when inserted into the holding unit 2 of the injection device 1 of the present invention, can be guided through its guide tube 10. In a preferred embodiment, the guide tube is in particular adapted in such a way that (i) an injection needle 11, when coupled to the injection device 1 of the present invention, and in particular to an syringe 14 when inserted into the holding unit 2 of the injection device 1 of the present invention, can be guided through its guide tube 10, and (ii) a catheter, when coupled to the injection device 1 of the present invention, can be guided through its guide tube (10), wherein most preferably the needle and the catheter run in parallel when both are coupled to the injection device 1 of the present invention and guided through the guide tube 10. Details of one embodiment of the needle guide 8 are shown in FIGS. 7, 8, 9 and 11. The needle guide 8 is preferably made of a metallic material, preferably of steel, more preferably of stainless steel, more preferably of implant grade steel and more preferably of an austenitic chromium-nickel-molybdenum steel, for example material 1.4044. The guide tube holder 9 has a recess through which the tubular guide tube 10 extends and the guide tube 10 is fixed to the guide tube holder 9 so that it does not change its position within the guide tube holder 9, but the guide tube 10 is rotatable along its longitudinal axis within the guide tube holder 9. In particular, the guide tube holder 9 and the guide tube 10 are toleranced to each other by means of a clearance fit so that the guide tube 10 is rotatable.

Preferably, the guide tube 10 comprises a channel 43 which lies outside the axis of symmetry of the guide tube 10, and wherein the guide tube 10 is adapted to guide the injection needle through said channel, i.e., the injection needle when coupled to the injection device 1 of the present invention, and in particular to an injection syringe 14 when inserted into the holding unit 2 of the injection device 1 of the present invention, is guided through that channel 43.

In one embodiment, the guide tube is assembled as shown in FIGS. 7, 8 and 9. In particular, the guide tube 10 consists in one embodiment of two parts, the large part (big guide) 25 and a small part (small guide) 26. Both of the counterparts are positively connected with each other and in particular, their sheath surfaces are polished together.

Assembly of the big guide 25 and the small guide 26 to the guide tube 10 can be performed by common techniques, for example by stacking or sliding one on top of the other.

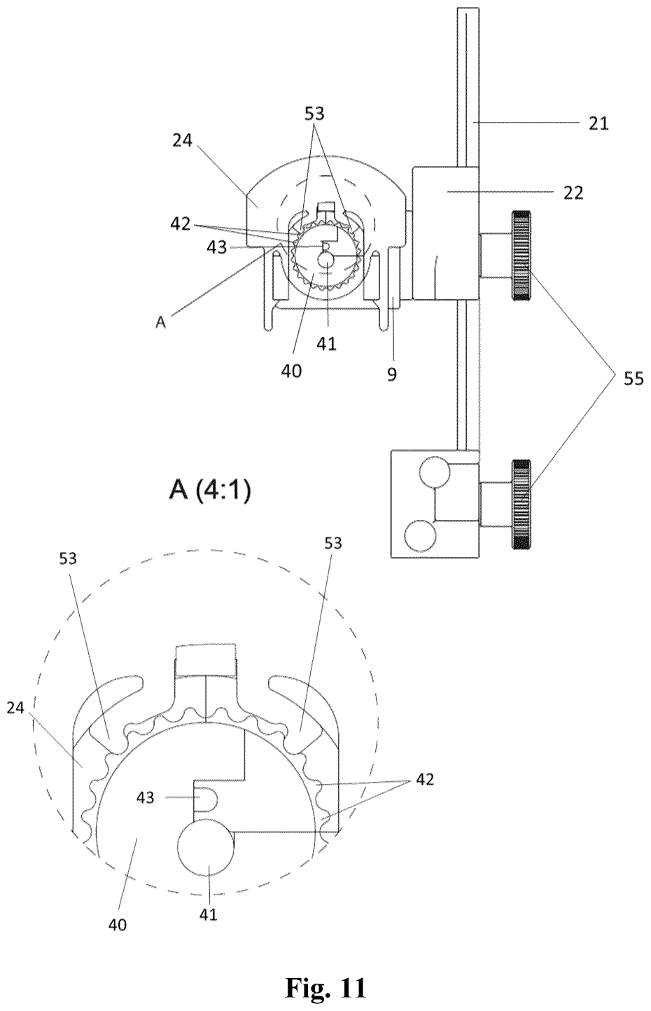

When the big guide 25 and the small guide 26 are assembled to the guide tube 10, a channel 43 is formed through which an injection needle can be guided as explained above. This channel 43 lies preferably outside the axis of symmetry of the guide tube 10; see for example FIG. 11.

In one embodiment, the guide tube 10 further comprises a channel 41 which preferably lies in the axis of symmetry of the guide tube 10, through which a catheter (not shown) can be guided. Accordingly, in one embodiment, when the big guide 25 and the small guide 26 are connected to the guide tube 10, two channels 43 and 41 are formed through which an injection needle and a catheter, respectively can be guided as explained above.

Thus, in one embodiment, at least the big guide 25 or the small guide 26, or the big guide 25 and the small guide 26 comprise(s) respective protrusion(s) to form the channel(s) 41 and/or 43 when assembled with the other part. In a preferred embodiment, the big guide 25 comprises a protrusion to form the catheter-channel 41 when assembled with the small guide 26 to form the guide tube 10 and the small guide 26 comprises a complementary protrusion to form the catheter-channel 41. In a further preferred embodiment, the small guide 26 further comprises an additional protrusion to form the needle-channel 43 when connected to the big guide 25 to form the guide tube 10.

In one embodiment, one section of the guide tube 10 comprises circumferential teeth 42 directed outwards with respect to the surface of the guide tube 10. In a preferred embodiment, the distance between two teeth is between 5° and 25°, preferably between 10° and 20° and most preferably 15°.

In one embodiment, the guide tube 10 comprises a handle 23 to rotate the guide tube 10 along is longitudinal axis. The handle 23 is preferably positioned perpendicular to the guide tube 10.

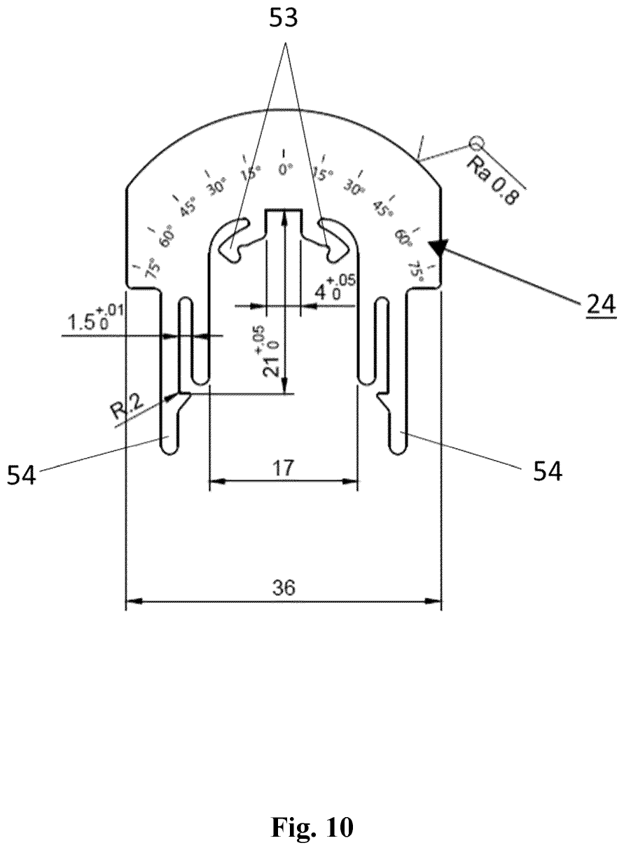

In one embodiment, the guide tube holder 9 of the injection device 1 of the present invention comprises a locking disk 24. One embodiment of the locking disk 24 is shown in FIG. 10. In particular, in one embodiment, the locking disk 24 is clamped onto the guide tube holder 9 from above so that the guide tube 10 is axially fixed in the guide tube holder 9. More particularly, the locking disk 24 is clamped via its grids i.e., clamp feet 54 onto the guide tube holder 9. In one embodiment, the locking disk 24 further comprises notches 53 in which the teeth 42 of the guide tube 10 can engage. This allows engagement of the teeth 42 of the guide tube 10 with the locking disk 24 when the guide tube 10 will be rotated by a certain distance. Thus, the guide tube 10 can have different locking positions in relation to the guide tube holder 9, wherein the distance between two locking positions, in particular two teeth 42 of the guide tube 10, is between 5° and 25°, preferably between 10° and 20° and most preferably 15°.

In one embodiment, the locking disk 24 has laser markers showing the different locking positions, e.g., from 0° to 90° in each direction, preferably from 0° to 75°.

The locking disk 24 is preferably made of a plastic material, more preferably of polyoxymethylene (POM).

Thus, when moving, in particular rotating the guide tube 10, the position of the needle 11 is changed by a certain distance, preferably between 5° and 25°, preferably between 10° and 20° and most preferably by 15°. For ease of handling, the guide tube 10 is in one embodiment operatively coupled to a handle 23, which can be used to move, in particular to rotate the guide tube.

Accordingly, movement of the guide tube 10 results in a change of the injection position. In a preferred embodiment, the injection positions are spaced apart from each other between 5°and 25°, preferably between 10°and 20°and most preferably by 15°, preferably wherein the injection positions extend along a crescent-shaped tissue in the organism. In one embodiment, the spacing of for example 15°between the at least two injection positions allows for targeting of radial sectors of the urethral sphincter muscle as injection sites. In particular, the targetable sphincter sectors are the superior 13 sectors of 15°each, spanning a total angle of 195°around the urethral axis, and preferably 9-10 sectors of 15°each, spanning a total angle of 150°around the urethral axis, and being centered symmetrically to the sagittal plane of the organism.

In one embodiment, the needle guide 8 is connected to a connection structure 12 of the injection device 1 of the present invention. The connection structure 12 operatively connects the above-described parts of the injection device 1 of the present invention. In particular, the connection structure 12 connects the holding unit 2 with the needle guide 8 and thus, also indirectly connects the actuating unit 3 with both mentioned components of the injection device 1, since the actuating unit 3 is mounted onto the holding unit 2. The connection structure can have any design as long as it connects the above-mentioned parts of the injection device 1 of the present invention. In one embodiment, the connection structure 12 comprises proximal elongated struts, preferably rods, preferably four elongated guide rods to which the holding unit 2 and the positioning structure 15 of the needle guide 8 of the injection device 1 of the present invention can be attached, i.e., removably connected.

In one embodiment, the connection structure 12 is made of a metallic material, preferably of steel, more preferably of stainless steel, more preferably of implant grade steel and more preferably of an austenitic chromium-nickel-molybdenum steel, for example material 1.4044.

In one embodiment, the connection structure 12 further comprises attachment means for an imaging device, in particular for the ultrasonic probe 27. The attachment means and the connection structure 12 are designed in a way that the imaging device 27 is removably couplable with the connection structure 12. One particular example of such a connection structure is the structure of BK Medical, Catalogue number UD0238.

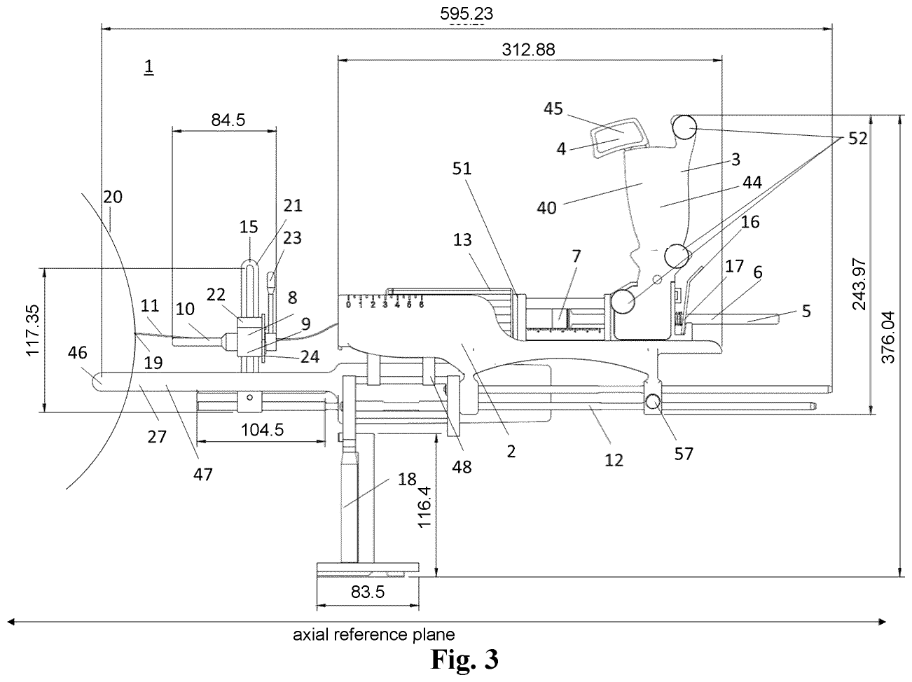

Accordingly, in one embodiment, the injection device 1 of the present invention further comprises an imaging device, preferably an ultrasound probe 27, which is attached to the injection device 1 via the above-described connection structure 12. One embodiment of the injective device 1 of the present invention which comprises said imaging device 27 is depicted in FIG. 3 and FIG. 4. In particular, the imaging device 27 extends in one embodiment in longitudinal direction and is arranged in parallel to the connection structure 12 and to the guide tube 10 when the guide tube 10 is perpendicular to the positioning structure 15, respectively, and below the guide tube 10 as shown in FIG. 3 and FIG. 4, wherein the head 46 of the ultrasound probe 27 is facing in the direction of the injection site 19 (and not in the direction of the actuating unit 3 of the injection device 1 of the present invention).

The ultrasound probe 27 is preferably a rod ultrasound probe, for example a vaginal or rectal ultrasound probe, preferably a vaginal ultrasound probe. In one embodiment, the shaft of the ultrasound probe 47 has a diameter of about 10 mm to 20 mm, preferably of about 15 mm to 17 mm, most preferably of 16 mm. In one embodiment, the ultrasound probe is of the type 8838 (Endocavity 3D 8838) of BK Medical. The ultrasound probe is preferably adapted for the generation of a radial and lateral-frontal sectioning pattern. In one embodiment, the ultrasound probe 27 can be moved axially in the connection structure 12, for example by losing and reconnecting of clamping rings 48 which fix the ultrasound probe 27 within the connection structure 12. In a manner known per se, the ultrasound probe 27 is connected to an ultrasound device (not shown) that can be used to acquire and display images of the tissue.

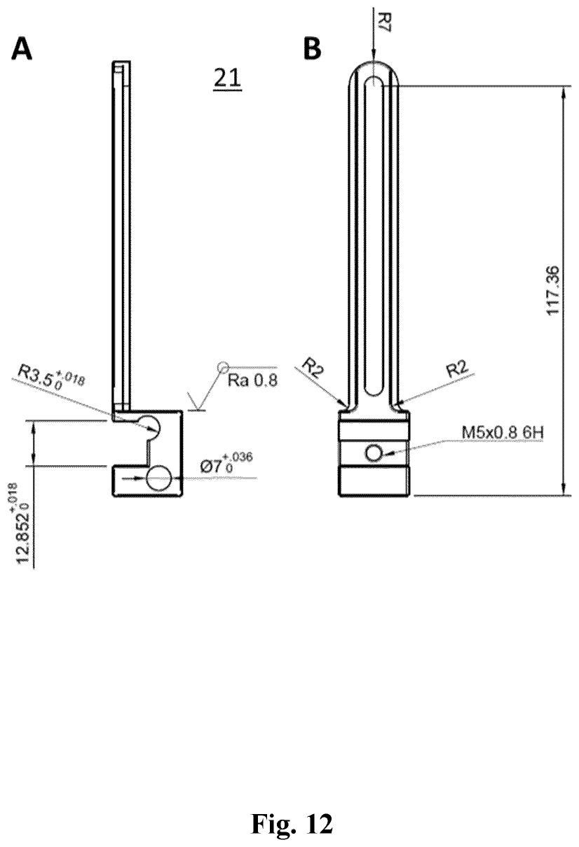

In one embodiment, the injection device 1 of the present invention further comprises a positioning structure 15, to which the needle guide 8 including the guide tube holder 9 and the guide tube 10 is connected. Details of one embodiment of the positioning structure are shown in FIGS. 11, 12 and 13. In one embodiment, the positioning structure 15 comprises at least a rail 21, wherein the rail 21 is moveably connected to the connection structure 12 of the injection device 1 of the present invention, preferably with knurled screws 55. In particular, the rail 21 extends in perpendicular direction to the connection structure 12, and the guide tube holder 9 is connected to the rail 21 so that the guide tube 10 extends in its functional orientation parallel to the connection structure 12, wherein the needle outlet opening 49 of the guide tube 10 faces towards the injection site 19. The rail in one embodiment is shown in FIG. 12.

Thus, in one embodiment, the positioning structure 15 including the rail 21 as well as the needle guide 8 including the guide tube 10 and the guide tube holder 9 is slidable along the connection structure 12 and in parallel to the imaging device, in particular in parallel to the shaft 47 of the longitudinal ultrasound probe 27 to adjust the distance of the guide tube 10 to the head 46 of the ultrasound probe 27.

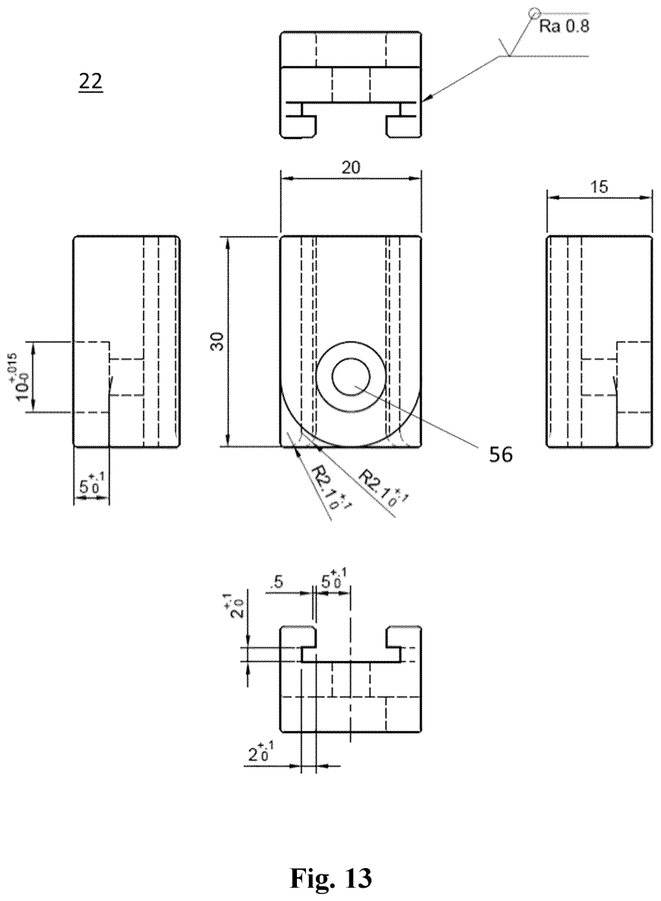

In a preferred embodiment, the positioning structure 15 further comprises a slide 22, wherein the needle guide 8 including the guide tube holder 9 and the guide tube 10 is connected to the above-described rail 21 via the slide 22, wherein the slide 22 is in one embodiment adapted to slide along the rail 21 in the above-mentioned directions. Alternatively, or preferably in addition, the slide 22 allows the needle guide 8 including the guide tube 10 and the guide tube holder 9 to pivot about its pivot axis. In particular, the slide 22 is pivotable towards the imaging device 27 and away, in particular towards the shaft 47 of the longitudinal ultrasound probe 27 and away, which is realized by a pivot bearing and the knurled screw 55 of the slide 22. In one embodiment, the slide 22 is pivotable by 360° about its pivot axis. A detailed view of the slide in one embodiment is given in FIG. 13 and one embodiment of the positioning structure 15 including the rail 21 and slide 22 is shown in FIG. 11.

The positioning structure 15 including the rail 21 and the slide 22 is made in one embodiment of a metallic material, preferably of steel, more preferably of stainless steel, more preferably of implant grade steel and more preferably of an austenitic chromium-nickel-molybdenum steel, for example material 1.4044.

The pivoting of the needle guide 8 and thus, of the guide tube 10 and consequently of the injections needle 11 when inserted into the injection device 1 of the present invention allows to adjust the injection angle.

The guide tube 10 fulfills different functions. On the one hand it provides a guidance for the injection needle 11 and on the other hand it sets the injection angle (angle of inclination of the injection syringe 11 relative to the axial reference direction). From the axial reference direction, the injection angle forms an angel in the range of 3° to 10°, preferably of 4° to 9°, more preferably of 5° or 7.5°, most preferably of 7.5°.

In one embodiment, the injection needle 11 is bent by 3° to 10°, preferably by 4° to 9°, more preferably by 5° or 7.5°, most preferably by 7.5°, i.e. its curvature is 3° to 10°, preferably 4° to 9°, more preferably 5° or 7.5°, most preferably 7.5°. In a preferred embodiment, the front part of the needle per se which protrudes the guide tube 10 has a curvature of 5°, and the guide tube 10 can be arranged to adjust the curvature of the needle, preferably by further 2.5°, i.e. the guide tube 10 can be adjusted upwards or downwards in relation to its longitudinal axis.

For safe guidance of the injection needle 11, the length of the tapering part of the guide tube 58, i.e., the part which comprises the needle outlet opening 49 and faces towards the injection site 19, is in general not restricted and is inter alia selected to ensure a specific maximum penetration depth of the injection needle 11 and/or to reach the respective tissue to be injected. For example, the female urethra is about 4.8 to 5.1 cm in length and the male urethra is about 20 to 25 cm in length. Accordingly, in one embodiment, the tapering part 58 has a length in the range of 2 cm to 50 cm, preferably of at least 4 cm, preferably in the range of 4 cm to 40 cm, preferably of 4 cm to 20 cm or 4 cm to 15 cm, but most preferably of about 4 cm.

The insertion depth of the injection needle 11 is selected depending on the desired application of the injection device 1 of the present invention. In general, the tissue in which the injectable substance is to be injected, for example a sphincter muscle, and in particular, the external urinary sphincter muscle, is observed with the imaging device 27 and when injecting the substance, the user of the injection device ensures that the injection needle 11 does not penetrate the tissue.

As mentioned above, the injection device 1 of the present invention comprises in one embodiment an injection needle 11, which is removable connected to the injection syringe 14 when inserted into the injection device 1, wherein the injection needle 11 protrudes from the needle outlet opening 49 of the guide tube 10 in bent form, wherein the curvature of the needle 11 is caused by a grinding 50 in the guide tube 10. In particular, the needle 11 has a curvature that is directed radially outwards relative to the axial orientation of the ultrasonic probe 27 and the guide tube 10, respectively.

In one embodiment, the injection needle 11 has a length between 10 cm and 30 cm, preferably between 15 cm and 25 cm and most preferably of 20 cm. In one embodiment, the injection needle 11 ranges in size from 18 to 23 gauge (G). In one embodiment, an injection needle 11 with the specifications 20 cm length and between 17 and 18 G is used.

In one embodiment, the injection device 1 of the present invention is connected to a mount, for example a bed or floor mount, preferably via a plinth 18 which is part of the connection structure 12.

As mentioned above, the present invention further relates to a method for injecting an injection substance into an organism, preferably into the external urinary sphincter muscle 20 (schematic, not drawn to scale), wherein the injection device 1 and the medical device, respectively, of the present invention is used comprising at least a step of a dosed injection of the injection substance by displacement of the lever 4 of the actuating unit 3 of the injection device 1 from first position 35 to a second position 36 as described in detail above.

In more detail and in a preferred embodiment, the method of the present invention comprises the following steps:
a) positioning of the injection device 1 of the present invention so that it has a predetermined position relative to the injection site 19 in the organism 20;
b) advancing the injection needle 11 until reaches the injection site 19, i.e., until the injection needle 11 penetrates the desired tissue 20;
c) injection of the injection substance via displacing the lever 4 of the actuating unit 3 of the injection device 1 from a first position 35 to a second position 36;
d) retraction of the needle 11; optionally,
e) changing the injection position by moving the guide tube 10 from a first locking position to a second locking position, preferably by moving the guide tube 10 clockwise by 15°;
   wherein the steps b) to e) can be repeated at different injection sites and/or until the entire injection substance is injected, or wherein only one injection is performed, or multiple injections at one injection site (steps a) to d), and repetition of steps b) to d), respectively). In a preferred embodiment, the steps b) to e) are repeated at least twice, preferably 2 to 13 times or to 18 times, more preferably 2 to 10 times or 2 to 15 times, and most preferably 9 or 10 times or 15 times, and wherein the injection is preferably performed at least two different injection sites, preferably at 2 to 13 different injection sites, more preferably at 2 to 10 different injection sites, and most preferably at 9 or 10 different injection sites. In one embodiment, 10 injections á 400 μl are performed at 10 different injection sites (low dose). In one embodiment, 9 injections á 400 μl are performed at 9 different injection sites (low dose). In another embodiment, 15 injections á 400 μl are performed at 9 or 10, preferably 10 different injection sites (high dose) meaning that at some injection positions two injections are performed.

The injection(s) can be located along the course of the urethra, for example midurethral, circumferential and/or proximal urethral injection(s) can be performed.

In one embodiment, 9 to 10 injections á 400 μl are performed, wherein one or two injections are performed in the central part of the external urinary sphincter muscle (−30° to +30° calculated from the centre of the longitudinally extending muscle), and each four injections á 400 μl are performed further radially outwards, preferably −45° to −90° and +45° to +90°. In another embodiment, 15 injections á 400 μl are performed, wherein one or two injections are performed in the venter part of the external urinary sphincter muscle (−30° to +30° calculated from the venter of the longitudinally extending muscle), and each four injections á 400 μl are performed further radially outwards, preferably −45° to −90° and +45° to +90° and in addition five further injections are performed in the above-mentioned central region (−30° to +30°, in particular at 0°, −15°, −30°, +15°, +30°).

The injection volume can also vary. For example, it can be adjusted in view of the amount of active ingredient in the injectable substance, for example in view of the amount of the density of the muscle-derived precursor cells to be injected for the treatment of the above-mentioned diseases. In particular, a lower or higher injection volume can be chosen. In case of the use of cells as injectable substance, the injections volume should preferably not exceed 500 μl and more preferably should not exceed 400 μl to ensure a sufficient supply of oxygen and to omit necrosis. However, even higher injection volumes are feasible as long as the supply of oxygen to the cells in ensured.

Accordingly, in one embodiment, any injection volume can be chosen as long as the supply of oxygen to the cells in ensured. For example, higher injections volumes, for example up to the ml range, can be chosen when the injectable substance spreads over a large area at the injections site, i.e., when the ratio of surface area to volume of the injection depot is large and thus oxygen can easily diffuse to the cells. In one preferred embodiment, the injection volume is less than or equal to 500 μl, preferably less than or equal to 400 μl, more preferably between 10 μl to 400 μl, more preferably between 50 μl and 400 μl, more preferably between 100 μl and 400 μl, more preferably between 150 μl and 400 μl, more preferably between 200 μl and 400 μl, more preferably between 250 μl and 400 μl, more preferably between 300 μl and 400 μl, more preferably between 350 μl and 400 μl and most preferably 400 μl.

Accordingly, the mentioned injection volumes can be administered once or multiple times as described above. In one embodiment, the total dose to be administered is between 10 μl and 10 ml, preferably between 1 ml and 10 ml, more preferably between 3 ml and 8 ml, more preferably between 4 ml and 6 ml, most preferably 4 ml or 6 ml.

The positioning step a) includes in a preferred embodiment the assembling of the injection device 1 of the present invention and the mounting of the device 1 onto a mount, for example a bed or floor mount, more particularly, a brachytherapy stepper arm, which is preferably positioned in front of the subject to be treated. In a preferred embodiment, the injection device 1 is also coupled to the imaging device, preferably to the ultrasound probe 27, via the attachments means of the connection structure 12, wherein the ultrasound probe 27 is preferably inserted vaginally for visualization of the bladder, the urethra and the external sphincter muscle.

The assembly step includes inter alia in a preferred embodiment the insertion of the injection syringe 14 including the injection substance, preferably together with the cooling unit 13, into the injection device 1, in particular into the holding unit 2 of the injection device 1, the placement of the injection needle 11 on the syringe nozzle 34, whereby the needle tip should be aligned with the 0° mark of the locking disk 24 from a distal view, and the guiding of the injection needle 11 through the small channel 43 which lies outside the axis of symmetry of the guide tube 10, wherein the handle 23 of guide tube 10 should preferably be directed vertically upwards. In a preferred embodiment, the assembly step further includes the guiding of the catheter through the central channel 41 of the guide tube 10, preferably wherein the catheter is inserted intravesically via the urethra and the bladder is filled with isotonic sodium chloride solution. In principal any catheter can be used which fulfills the above-described function, but in a preferred embodiment the catheter has a length 30 to 50 cm, preferably of 35 to 45 cm, more preferably of about 40 cm, in particular of 41 cm. One particular example of a catheter that can be used in accordance with the present invention is a SupraCath single catheter, 3 ml, Ch. 8, 41 cm long.

The positioning step a) further includes in a preferred embodiment the filling of the needle 11 with the injection substance by pulling the lever 4 (one or several times) until the first drops of the injection substance emerge at the tip of the needle 11.

The injection needle 11 is preferably advanced until the needle tip just emerges from the guide tube 10 and is preferably seen in the ultrasound image, which is preferably performed by advancing the holding unit 2 including the actuating unit 3 along the connection structure 12.

Step b), i.e., advancing the injection needle 11, is preferably performed by advancing the actuating unit 3 including the holding unit 2 of the injection device 1 of the present invention along the connecting structure 12 in direction of the injection site 19, until the injection needle 11 reaches the injection site 19, wherein the operator, e.g. the physician, is preferably guided by the ultrasound image.

The injection step c) results in a dosed injection of preferably 400 μl of the injection substance via the mechanisms as described above. In particular, the lever 4 of the actuating unit 3 is triggered and dependent on the dose to be injected, the lever 4 is triggered once or twice.

The retracting step d) comprises preferably the complete retraction of the needle 11 into the guide tube 10. The step of retracting the syringe (step d)) is preferably performed simultaneously with the actuating of the lever and is preferably also performed manually by the operator by retracting the actuating unit including the holding unit along the connection structure, wherein the operator is again preferably guided by the ultrasound image.

Step e) preferably further includes the rotation of the image plane of the ultrasound probe 27 by at least one increment.

The following describes the preferred use of the invention in the treatment of a muscle dysfunction, preferably a sphincter muscle dysfunction, preferably a dysfunction of the external urinary sphincter and thus, preferably in the treatment of urinary incontinence, preferably female stress urinary incontinence (SUI). The injection device 1 according to the invention is preferably used for the treatment of said indications with muscle precursor cells. By injecting cultured muscle precursor cells into the corresponding muscle, muscle function is restored. In particular, by injecting the cells into the urethral sphincter muscle, the contractile force is increased and thus continence is restored. When treatment is particularly preferred performed with autologous cells, the following steps are provided:
a) obtaining a tissue sample by a skeletal muscle biopsy of the subject to be treated;
b) isolation of muscle precursor cells, preferably by surgically removing fat-, and/or tendon-, and/or connective tissue from the tissue sample; mincing and enzymatic digestion of the tissue sample; reducing the number of fibroblast cells, thereby yielding a population of muscle precursor cells;
c) expansion of the muscle precursor cells;
d) formation of the injection substance, preferably by mixing the cells with a carrier solution, like a collagen solution, preferably wherein the concentration of collagen is 1-4 mg/ml, more preferably about 2 mg/ml; and
e) injecting the injection substance into the corresponding muscle, preferably the sphincter muscle, preferably the external urethra sphincter muscle.

The muscle precursor cells are preferably provided as described in WO 2019/215090 A1 which content is herein incorporated by reference.

After the formation of the injection substance and before injection, said injection substance is filled into the syringe 14.

Alternatively, the formation of the injection substance can be performed by a syringe with two syringe chambers, wherein one chamber comprises the muscle precursor cells and the other chamber the carrier solution, like the above-mentioned collagen solution. During simultaneous injection from both syringe chambers, the carrier is mixed with the cells and thus, forms the injection substance.

The syringe 14 filled with the injection substance is inserted into the injections device 1 of the present invention and injection is performed as described above.

One detailed example how to assemble and use the injection device and the medical device of the present invention for the treatment of female urinary incontinence is provided in the following:
1) The cooling tool 13 including cell suspension in the syringe 14 is removed from the transport container and placed on sterile ice until use.
2) The needle guide 8 including guide tube 10 and guide tube holder 9 as well as the positioning structure 15 are placed on sterile ice.

### Preparation of the Connection Structure 12:

3) The injection needle 11 is unpacked; the protective cover, the blunt guide needle and the inner mandrel are removed. The injection needle is placed on sterile ice.
4) The floor/bed mount (brachystepper) is positioned in front of the patient 20.
5) The connection structure 12 is connected with the plinth 18 onto the brachystepper and positioned.
6) A sterile cover is placed around the plinth 18 over the brachystepper.
7) The ultrasonic probe 27 is treated with lubricant, provided with protection and treated again with lubricant. Subsequently, the ultrasonic probe 27 is inserted into the holder on the connection structure 12 (observe the pin and notch) and fixed with the two clamping rings 48.
8) The holding unit 2 is pushed onto the four proximal guide rods of the connecting structure 12 until the rear feet are flush with the ends of the guides; if necessary, the holding unit 2 is temporarily fixed with the two clamping screws 57.
9) The positioning structure 15 (including guide tube holder 9) including pivot bearing is pushed onto the two distal guide rods of the connecting structure 12 until it is approximately in the central position and temporarily fixed with the clamping screw 55.

### Adjusting of the Ultrasound Probe and the Needle Guide:

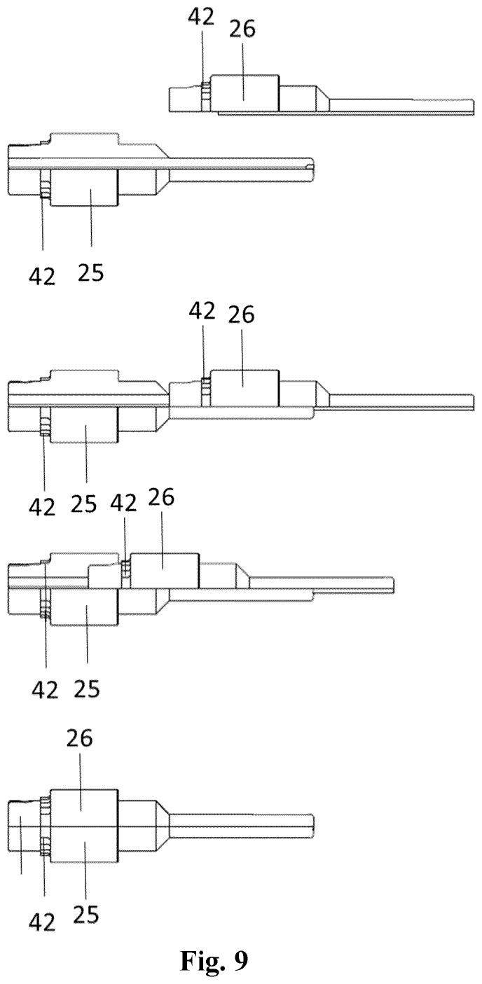

10) General anesthesia by intubation.
11) Antibiotic prophylaxis with Zinacef 1.5 g i.v. (alternative antibiotic will be determined preoperatively in case of intolerance).
12) Lithotomy storage
13) Disinfection and covering of the external genitalia and meatus urethrae externus.
14) By moving the brachystepper, the ultrasound probe 27 is inserted vaginally for visualization of bladder with bladder neck, rhabdomyosphincter and urethra in 3D mode, after which the position of the brachystepper is fixed again.
15) The Charriere 8 Balloon indwelling catheter is removed from the packaging and threaded through the needle guide 8, in particular through the central channel 41.
16) The catheter is inserted intravesically via the urethra; block with 3 ml glyco-block or NaCl solution. Urinary bladder is emptied.
17) Using a bladder syringe, the urinary bladder is filled with 100 ml NaCl; a Kocher clamp is attached to the end of the catheter. Extracorporeal parts of the now clamped catheter are temporarily positioned on the holding unit 2.
18) The distal section of the indwelling catheter (behind the needle guide 8) is inserted into the central channel of the big part of the guide tube (¾ guide) 25, 10.
19) The smaller part of the guide tube (¼ guide) 26, 10 is pushed onto the big part (¾ guide) 25, 10 from the front so that the guide tube 10 surrounds the catheter; see also FIG. 9.
20) The guide tube 10 is inserted distally into the guide tube holder 9.
21) The plastic spring, i.e., the locking disk 24 is clamped onto the guide tube holder 9 from above so that the guide tube 10 is axially fixed in the guide tube holder 9. It is important to ensure that the clamp feet 54 are oriented straight=unbent—accompanied by a click noise.
22) Correct clamping is checked by rotating the guide tube 10 with the handle of the guide tube 23, whereby the guide tube 10 should engage in the 15° increments 42 provided. The guide tube 10 is then returned to the 0° position (handle of the guide tube 23 pointing upwards). An angle degree is printed on the plastic cord of the locking disk 24.
23) The two clamping screws 55 of the needle guide positioning structure 15 are loosened so that the guide tube 10 can be inserted urethrally along the balloon catheter.
24) In accordance with the correct position and alignment, the clamping screws 55 of the needle guide 10 are tightened; special attention is paid to the sound-erasing abdominal space of the guide tube 10, the distance to the bladder neck (DK balloon) and a perfect view of the rhabdomyosphincter (hypoechogenic, periurethral structure).

### Preparation of the Actuating Unit (Dosing Tool) 3:

25) The cover of the lever shell 44 is screwed onto the lever shell 32.
26) The locking spring (retaining element 16) is held down so that the piston rod 6 can be pushed back completely,
27) after which the actuation and complete return of the trigger (lever 4) is checked. For this purpose, the lever 4 is operated five times, after which the piston rod 6 should have moved forward.
28) The cooling tool 13 including the cell suspension is taken from the sterile ice and mixed by turning.
29) The cooling tool 13 including the syringe 14 is screwed onto the actuating unit 3.
30) The luer lock closure of the syringe 14 is removed.
31) The needle 11 is screwed onto the Luer lock nozzle 34 of the syringe 14, whereby the needle tip must be aligned with the 0° mark (12 o'clock) from the distal view.
32) The actuating unit 3 is inserted a short distance into the holding unit 2 with the handle of the lever 45 facing upwards.
33) It is checked again that the needle tip is aligned with the 0° mark (12 o'clock) from a distal view. By slightly loosening the cooling unit thread 51 and turning the syringe 14, the orientation of the needle 11 cut can be adjusted; then the actuating unit 3 is tightened again and inserted into the holding unit 2.
34) The needle 11 is filled with liquid (priming) by pulling the trigger 4 (possibly several times), only until the first drops emerge at the tip of the needle 11.
35) The actuating unit 3 is advanced in the holding unit 2 only so far that the distance to the front limit in the holding unit 2 is at least as great as the expected puncture distance.
36) The clamping screws 57 of the holding unit 2 are loosened; then the holding unit 2 together with the actuating unit 3 is carefully pushed forward on the guide rods of the connection structure 12, with the needle 11 tip being manually inserted into the upper lumen 43 in the guide tube 10.
37) The holding unit 3 including the actuating unit 3 is carefully pushed further forward until the needle 11 tip just emerges from the guide tube 10 and is visible in the ultrasound image; the clamping screws 57 are tightened.

### Injection of the Cell (Muscle Derived Precursor Cells) Suspension:

38) The starting position is selected from a distal view at 0° (12 o'clock); the handle of the guide tube 23 and the handle of the lever 45 of the actuating unit 3 point vertically upwards.
39) The actuating unit 3 is manually advanced, observing the ultrasound image, until the needle 11 tip penetrates sufficiently far into the rhabdomyosphincter 20.
40) The therapy product is injected by actuating the trigger 4 with the index finger. Depending on the dosage, the trigger 4 is actuated once or twice. One actuation of the trigger 4 corresponds to the volume of 0.4 ml. The volume present in the syringe 14 can be checked using the scale printed on the holding unit 2.
41) When the needle 11 is fully retracted into the guide tube 10, turning the guide tube 10 clockwise by 1 click (15°) and tilting the actuating unit 3 (15°) sets the next injection level.
42) For each click with the guide tube 10, the image plane of the ultrasound probe 27 is also rotated by at least one increment (+/−key).
43) Radially, further injections are performed in a clockwise direction (for example +15°, +30°, +45°, +60°), with steps 39-42 being repeated in each case.
44) When the needle 11 is fully retracted into the guide tube 10, realign the guide tube 10 and the actuating unit 3 to 12 o'clock (0°) from the distal view and place the remaining injections using an analogous counterclockwise procedure (for example, positions −15°, −30°, −45°, −60°).
45) To achieve the most complete coverage of the rhabdomyosphincter, a total of:
   Low Dose 4 ml=9-10 injections per 0.4 ml performed (1-2× central, 4× each right and 4× left).
   High Dose 6 ml=15 injections per 0.4 ml. Same procedure followed by a second injection in the central positions (range from −30°to +30°). The dorsal region periurethral is omitted.
46) After the last injection, the actuating unit 3 including the needle 11 is completely withdrawn and put aside.
47) The catheter is unblocked.
48) The clamping screws 55 on the needle guide positioning structure 15 are loosened so that the guide tube 10 can be retracted.
49) The Charr 8 catheter is removed from the body. If the catheter cannot subsequently be pulled out through the lumen in the needle guide 41, the guide tube holder 9 must be pulled out by removing the locking disk 24 and disassembled to expose the catheter.
50) Brachystepper arm (Floor/Bed mount) with connection structure 12 and ultrasound probe 27 is retracted so that the probe 27 is no longer in the body.
51) Insertion of a new balloon catheter Charr. 12 and block with 10 ml Glycoblock or NaCl.
52) End of the procedure
53) Disassembly of the injection device 1.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

The features of the invention disclosed in the description, the drawings and the claims may be of importance, individually or in combination, for the realization of the invention in its various embodiments.

### LIST OF REFERENCE SIGNS

1 injection device
2 holding unit
3 actuating unit
4 lever (trigger)
5 piston
6 piston rod
7 piston stop
8 needle guide
9 guide tube holder
10 guide tube (consisting of two parts)
11 injection needle
12 connection structure
13 cooling tool
14 syringe
15 positioning structure
16 retaining element (clamping plate)
17 first compression spring
18 plinth
19 injection site
20 organism, in particular sphincter muscle of the organism
21 rail
22 slide
23 handle of the guide tube 10
24 locking disk
25 big part of the guide tube 10
26 small part of the guide tube 10
27 imaging device, in particular ultrasound probe
28 plunger of the syringe 14
29 piston of the syringe 14
30 second compression spring
31 actuator (clamping piece)
32 lever shell
33 bolt connecting the actuator 31 (clamping piece) and the lever 4 (trigger)
34 nozzle of the syringe 14
35 first position of the lever 4
36 second position of the lever 4
37 first position of the actuator 3 (clamping piece)
38 second position of the actuator 3 (clamping piece)
39 syringe body
40 housing of the lever 4
41 catheter channel
42 radial teeth
43 injection needle channel
44 cover of the lever shell 32
45 handle of the lever 4
46 head of the ultrasound probe 27
47 shaft of the ultrasound probe 27
48 clamping rings
49 needle outlet opening
50 grinding
51 screw thread
52 knurled screws of the actuating unit 3
53 notches in which the teeth 42 of the guide tube 10 can engage
54 grids for attaching to the guide tube holder 9, i.e. clamp feet
55 knurled screws of the positioning structure 15
56 opening for the knurled screw 52
57 clamping screws
58 tapering part of the guide tube
59 stop of the housing
60 facets of the cooling tool

1. An injection device which is provided for injection of an injectable substance with a syringe into an organism, comprising:
a holding unit, which is adapted to hold the syringe;
an actuating unit, which is adapted for dosed injection of the injectable substance and which is removably coupled to the holding unit and couplable with a plunger of the syringe when the holding unit holds the syringe, preferably wherein the actuating unit comprises a lever and a piston, wherein a displacement of the lever from a first position to a second position results in a movement of the piston along its longitudinal axis, wherein the actuating unit is adapted to transfer the movement at least partly to the plunger of the syringe when the syringe is coupled with the actuating unit;
a needle guide that is adapted to be removably couplable with an injection needle, and that is adapted for changing the injection position of the injection needle; and
a connection structure which connects the holding unit and the needle guide.

2. The injection device according to claim 1, wherein the needle guide is adapted to be removably couplable with a catheter, preferably a transurethral bladder catheter.

3. The injection device according to claim 1, wherein the needle guide comprises a guide tube and a guide tube holder, wherein the guide tube is rotatable along its longitudinal axis within the guide tube holder.

4. The injection device according to claim 3, wherein the guide tube is composed of two parts, a large part and a small part, which when assembled, form the guide tube.

5. The injection device according to claim 4, wherein when the large part and the small part of the guide tube are assembled to the guide tube, at least one channel is formed, preferably wherein two channels and are formed, preferably wherein the one channel lies outside the axis of symmetry of the guide tube and is used to guide the injection needle through and wherein the other channel lies within the axis of symmetry of the guide tube and is used to guide the catheter through.

6. The injection device according to claim 5, wherein the needle guide is adapted in such a way that the injection needle and the catheter, when both are coupled to the needle guide, run in parallel through the guide tube and the channels respectively.

7. The injection device according to claim 1, wherein the injection device further comprises a cooling tool, which is hold by the holding unit, preferably wherein the cooling tool is removable and/or wherein the cooling tool is rotatable about its longitudinal axis.

8. The injection device according to claim 7, wherein the cooling tool comprises facets, which are spaced apart from each other by 10° to 20° C., preferably by 15°, and which preferably run around the cooling tool.

9. The injection device according to claim 1, wherein the actuating unit further comprises a retaining element, preferably a retaining bracket, wherein the retaining element frictionally engages the rod of the piston in a retaining position in such a way that the piston is held in position and prevents the piston from being retracted along its longitudinal axis when the lever moves from the second position to the first position.

10. The injection device according to claim 1, wherein a displacement distance of the lever between the first position and the second position is between 17 mm and 23 mm, preferably between 19 mm and 21 mm, and most preferably 20 mm.

11. The injection device according to claim 1, wherein the injection device is adapted to guide the injection needle to at least two injection positions, the location of which differ from each other, by rotation of a guide tube, wherein the guide tube comprises a channel which lies outside the axis of symmetry of the guide tube, and wherein the guide tube is adapted to guide the injection needle through said channel.

12. The injection device according to claim 3, wherein the guide tube comprises circumferential teeth directed outwards with respect to the surface of the guide tube, wherein when the guide tube is rotated within the guide tube holder, the teeth engage with a locking disk, which is arranged on the guide tube holder, preferably wherein the distance between two teeth of the guide tube is 15°.

13. The injection device according to claim 1, wherein the injection positions are spaced apart from each other by 10° to 20°C., preferably by 15°, preferably wherein the injection positions span a total angle of 195°, preferably of 150° around the urethral axis and are centered symmetrically to the sagittal plane.

14. The injection device according to claim 1, wherein a guide tube comprises a channel which lies inside the axis of symmetry of the guide tube, and wherein the guide tube is adapted to guide a catheter through said channel.

15. The injection device according to claim 1, wherein the connection structure is adapted to be removably couplable with an imaging device, preferably an ultrasonic probe.

16. The injection device according to claim 1, which further comprises an imaging device, preferably a longitudinal ultrasonic probe, which is removably coupled to the injection device via the connection unit.

17. The injection device according to claim 1, wherein the needle guide is movable, preferably wherein
(i) the needle guide is movable in relation to an imaging device, preferably wherein the distance of the needle guide to a shaft of the imaging device is adjustable;
(ii) the needle guide is movable in relation to the holding unit, preferably wherein the distance of the needle guide to the holding unit is adjustable and wherein the distance of the needle guide to the shaft of the imaging device remains the same; and/or
(iii) the needle guide is pivotable.

18. The injection device defined by claim 1, and wherein at least a front part of the injection needle which protrudes from a guide tube towards the injection site has a curvature that is directed radially outwards relative to an axial orientation of an ultrasonic probe and the guide tube, respectively, preferably wherein the injection needle comprises a curvature of 3° to 10°, preferably of 5° to 7.5°, and more preferably of 5° to less than 7.5°.

19. Injection device, injection needle and catheter, wherein the injection device and the needle are defined by claim 1, and wherein the catheter is preferably a transurethral bladder catheter.

20. Injection device as illustrated in FIGS. 1 to 13 as well as equivalents thereof.

21. Medical device which is adapted for injection of an injectable substance into an organism, comprising the injection device according to claim 1, and a syringe with a syringe body and a piston, and an injection needle, wherein the syringe is arranged in the holding unit of the injection device, and/or wherein the injection needle is arranged in the needle guide, preferably wherein the medical device further comprises an imaging device, preferably an ultrasonic probe, preferably wherein the medical device further comprises a cooling tool as defined in claim 8 or 9, preferably wherein the medical device further comprises a catheter which is arranged in the needle guide.

22. Use of a syringe comprising an injectable substance in the injection device according to claim 1 or in the medical device according to claim 21.
